(19) United States
(12) Patent Application Publication
(10) Pub. No.: US 2011/0022164 A1
(43) Pub. Date: Jan. 27, 2011

(54) MEDICAL DEVICE FOR USE IN TREATMENT OF A VALVE

(75) Inventors: Martin QUINN, Blackrock (IE); Eamon Brady, Elphin (IE)

Correspondence Address: SUGHRUE MION, PLLC, 2100 PENNSYLVANIA AVENUE, N.W., SUITE 800, WASHINGTON, DC, 20037, US

(73) Assignee: MEDNUA LIMITED, Dublin (IE)

(21) Appl. No.: 12/709,251

(22) Filed: Feb. 19, 2010

Related U.S. Application Data
(63) Continuation-in-part of application No. PCT/IE2008/000104, filed on Oct. 20, 2008.
Application No. PCT/IE2008/000104 is a continuation-in-part of application No. 11/763,590, filed on Jun. 15, 2007.
Application No. 11/763,590 is a continuation-in-part of application No. 11/300,580, filed on Dec. 15, 2005.
(60) Provisional application No. 60/982,799, filed on Oct. 26, 2007.
(60) Provisional application No. 60/813,694, filed on Jun. 15, 2006.

(30) Foreign Application Priority Data
Dec. 15, 2004 (IE) S2004/0841

(51) Int. Cl. A61F 2/24

(52) U.S. Cl. 623/2.11; 623/2.36

(57) ABSTRACT

A medical device 1 for use in treatment of a valve comprises a treatment element 2 which is configured for location at a region of coaption of leaflets 3 of a valve to resist fluid flow in a retrograde direction through an opening 7 of the valve. The device also comprises a support 4 for the treatment element 2, and an anchor 8 for anchoring the support 4 to a heart wall. The treatment element 2 and/or at least a part of the support 4 comprises a hydrogel.

### INTRODUCTION

This invention relates to a medical device suitable for use in treatment of a valve, and to a method of treating a valve.

The heart contains four valves, two semilunar, the aortic and pulmonary valves, and two atrioventricular (AV) valves, the mitral and tricuspid valves. The heart fills with blood from the lungs and body when the AV valves are open. When the heart pumps or contracts, the AV valves close and prevent the blood from regurgitating backwards. The semilunar valves open when the heart pumps allowing the blood to flow into the aorta and main pulmonary artery.

Dysfunction of the cardiac AV valves is common and may have profound clinical consequences. Failure of the AV valves to prevent regurgitation leads to an increase in the pressure of blood in the lungs or liver and reduces forward blood flow. Valvular dysfunction either results from a defect in the valve leaflet or supporting structure, or dilation of the fibrous ring supporting the valve. These factors lead to a failure of valve leaflets to meet one another, known as co-aptation, allowing the blood to travel in the wrong direction.

This invention is aimed at providing a medical device which addresses at least some of these problems.

### STATEMENTS OF INVENTION

### Section 1

According to the invention there is provided a medical device suitable for use in treatment of a valve, the device comprising a treatment element configured to be located at the region of co-aptation of leaflets of a valve to resist fluid flow in a retrograde direction through an opening of the valve, the treatment element being movable between a first treatment configuration and a second treatment configuration.

In one embodiment of the invention the treatment element is movable in a plane substantially perpendicular to a longitudinal axis extending through an opening of a valve. Preferably in the second treatment configuration, the shape of the treatment element approximates the shape of an opening of a valve. Ideally in the second treatment configuration, the treatment element is configured to substantially fill an opening of a valve. The treatment element may have a substantially crescent-shape in lateral cross-section in the second treatment configuration. The treatment element may be particularly suitable for treating a mitral valve in a heart which has a crescent-shaped opening. The treatment element may have a substantially oval-shape in lateral cross-section in the second treatment configuration. The treatment element may have a substantially circular-shape in lateral cross-section in the first treatment configuration.

In one case the treatment element is movable from the first treatment configuration to the second treatment configuration upon engagement of leaflets of a valve with the treatment element.

In another embodiment the treatment element is movable from a storage configuration to the first treatment configuration. Preferably the treatment element is movable from the storage configuration to the first treatment configuration upon contact of a fluid with the treatment element. Ideally the treatment element is movable from the storage configuration to the first treatment configuration upon contact of blood with the treatment element. Most preferably the treatment element is expandable from the storage configuration to the first treatment configuration. The treatment element may comprise a porous material. The treatment element may comprise a hydrogel material. Preferably the treatment element comprises one or more beads of a hydrogel material.

In another case the treatment element comprises a hollow interior space. Preferably the interior space is enclosed.

In one embodiment the treatment element comprises a shape-memory material.

In one case the device comprises a reinforcement element to reinforce the treatment element. Preferably at least in the storage configuration, the reinforcement element is located around at least part of the treatment element. Ideally at least in the treatment configuration, at least part of the reinforcement element is embedded within the treatment element. The reinforcement element may comprise a braid material. The reinforcement element may comprise a fibre material.

In another embodiment the device comprises at least one support element to support the treatment element at the region of co-aptation of leaflets of a valve. Preferably the device comprises at least one anchor element to anchor the support element to a wall of body tissue. Ideally the anchor element is located at the distal end of the support element. Most preferably the proximal end of the support element is unconstrained relative to a wall of body tissue.

In another case the device comprises a delivery member coupleable to the treatment element to facilitate delivery of the treatment element to the region of co-aptation of leaflets of a valve. Preferably the delivery member comprises a delivery catheter for housing at least part of the treatment element.

In another aspect of the invention there is provided a method of treating a valve, the method comprising the step of locating a treatment element at the region of co-aptation of leaflets of the valve to resist fluid flow in a retrograde direction through an opening of the valve, the treatment element moving between a first treatment configuration and a second treatment configuration.

In one embodiment of the invention in the second treatment configuration, the treatment element fills the valve opening.

In one case the treatment element is moved from the first treatment configuration to the second treatment configuration upon engagement of the valve leaflets with the treatment element.

In another embodiment the treatment element moves from a storage configuration to the first treatment configuration. Preferably the treatment element moves from the storage configuration to the first treatment configuration upon contact of a fluid with the treatment element. Ideally the treatment element moves from the storage configuration to the first treatment configuration upon contact of blood with the treatment element. Most preferably the treatment element expands from the storage configuration to the first treatment configuration.

In another case the method comprises the step of delivering the treatment element to the region of co-aptation of the valve leaflets. Preferably the method comprises the step of supporting the treatment element at the region of co-aptation of the valve leaflets. Ideally the method comprises the step of anchoring the treatment element to a wall of body tissue.

Other features are described in the following sections and these are incorporated in their entirety in this section also.

### Section 2

According to the invention there is provided a medical device suitable for use in treatment of a valve, the device comprising; a treatment element which is configured for location at a region of coaption of leaflets of a valve to resist fluid flow in a retrograde direction through an opening of the valve; a support for the treatment element; and an anchor for anchoring the support to a heart wall wherein the treatment element and/or at least part of the support comprises a hydrogel.

In one embodiment the treatment element is supported at the region of coaptation by the support element. The treatment element has a collapsed delivery configuration and an expanded treatment configuration. The treatment element has a dehydrated state and a hydrated state and the dehydrated state corresponds to the collapsed delivery configuration and the hydrated state corresponds to the expanded treatment configuration.

In the hydrated state the hydrogel comprises primarily of a polymer and a hydrating liquid and at least some of the polymer molecules are cross-linked. The hydrating liquid comprises saline, contrast media, a biocompatible fluid, silicone fluid, or blood plasma components. In the expanded hydrated state the hydrogel is mostly liquid with solid polymer network spanning the occupied volume. Preferably the hydrogel comprises at least 50% liquid by volume in the hydrated state. Preferably the hydrogel comprises at least 70% liquid by volume in the expanded hydrated state. More preferably the hydrogel comprises at least 80% liquid by volume in the expanded hydrated state. More preferably the hydrogel comprises at least 90% liquid by volume in the expanded hydrated state. More preferably the hydrogel comprises at least 95% liquid by volume in the expanded hydrated state. More preferably the hydrogel comprises from 95% to 99% liquid by volume in the expanded hydrated state.

The density of the treatment element is in the range 1200 kg/m³ to 1025 kg/m³ in the expanded state. In one embodiment the polymer network of the hydrogel is at least partially composed of a synthetic polymer, a protein or a natural polymer. In one case the compliance of the treatment element in the expanded hydrated state is greater than the compliance of the treatment element in the collapsed state.

In one embodiment the polymer network of the hydrogel is loaded with an active compound which is eluted from the hydrogel over time. The eluted compound is selected from one or more of an anticoagulant, an anti-thrombin, an anti-platelet, an agent to prevent thrombosis, an anti-proliferative, an anti-fibrotic, an agent to promote endothelialisation, and a drug. The eluted compound comprises heparin or a factor Xa inhibitor.

In another embodiment the hydrogel is porous.

In another embodiment the hydrogel is at least partially solid. The hydrogel comprises hydrophilic chain segments. The hydrophilic chain segments comprise high electronegativity atoms and said high electronegativity atoms comprise at least 10% of the atomic mass of the chain segment. Preferably the hydrophilic chain segments comprise high electronegativity atoms and said high electronegativity atoms comprise at least 20% of the atomic mass of the chain segment. Preferably the hydrophilic chain segments comprise high electronegativity atoms and said high electronegativity atoms comprise at least 25% of the atomic mass of the chain segment. Preferably the hydrophilic chain segments comprise high electronegativity atoms and said high electronegativity atoms comprise at least 30% of the atomic mass of the chain segment.

The polymer of the hydrogel is preferably a hydrocarbon polymer with a high concentration of hydrophilic groups. The hydrogel may be selected from one or more of polyvinyl alcohol (PVA), sodium polyacrylate, hydrophilic acrylate polymers, hydrophilic polymethacrylates, 2-hydroxyethyl-methacrylate (HEMA), ethylene glycol bismethacrylate, hyluronan polymers, poly(anhydride esters), poly(vinylpyrroldine), poly(ethyloxazoline), poly(ethylene glycol)-co-poly(propylene glycol) block copolymers, hydrophilic methacrylamides, and a polyethylene glycol based polyurethane.

In another embodiment the treatment element is sonolucent. This allows the treatment element to be contrasted with surrounding tissue using echocardiography.

In one embodiment the hydrating liquid at least comprises a contrast medium.

This section 2 describes aspects of anchoring treatment elements to body structures. Additional features and embodiments are described in more detail in Sections 3, 6, 7 and 10 of this statement and are incorporated in their entirety in this section 2 by reference.

This section 2 describes aspects of mounting treatment elements at the treatment location. Additional features and embodiments are described in more detail in Sections 3, 6, 7 and 10 of this statement and are incorporated in their entirety in this section 2 by reference.

This section 2 describes aspects of delivering the treatment elements. Additional features and embodiments are described in more detail in Section 4 of this statement and are incorporated in their entirety in this section 2 by reference.

This section 2 describes the design construction and materials of treatment elements of the devices. Additional features and embodiments are described in more detail in Sections 1, 5 and 9 of this statement and are incorporated in their entirety in this section 2 by reference.

This section 2 describes methods of use of the treatment elements. Additional features and embodiments are described in more detail in Section 8 and are incorporated in their entirety in this section 2 by reference.

### Section 3

According to the invention there is provided a medical device suitable for use in treatment of a valve, the device comprising a treatment element, a support and a mounting tube wherein, the treatment element is configured to be located at the region of co-aptation of leaflets of a valve to resist fluid flow in a retrograde direction through an opening of the valve, the support is configured to anchor the treatment element to a tissue wall, the treatment element being mounted to at least one mounting tube.

In one embodiment the at least one mounting tube is rotatable relative to the support. The support extends from the tissue wall and supports the treatment element at the region of coaptation. In one aspect the treatment element is sealingly mounted to the at least one mounting tube. In another aspect the at least one mounting tube is integral with the treatment element. The mounting tube may comprise an extruded tube. The material of the mounting tube may comprise a polymer, or a metal. Preferably the material of the mounting is a biocompatible polymer or metal. The material of the mounting tube may comprise a polyurethane, a polyether polyurethane, a polycarbonate polyurethane, a polydimethysiloxane polyurethane, a silicone, a fluoropolymer, a polyester, polyethylene terephthalate, polyethylene naphthalate, a polyolefin, a polyethylene, ultra high molecular weight polyethylene, polyetheretherketone (PEEK), polyether ketone (PEK), stainless steel, a stainless alloy, a super elastic metal, a shape memory metal, and a nitinol.

In another embodiment the mounting of the treatment element on the mounting tube comprises an interface layer. In one variant the interface layer comprises a mixture of mounting tube material and treatment element material. The interface layer is at least partially resistant to fluid flow. In one embodiment the interface layer is formed in a process that involves the local flow of polymer of at least one of the treatment element or the mounting tube. The local flow of polymer may be created in a welding process or a solvent bonding process.

In one embodiment the support extends through the treatment element and the at least one mounting tube encircles the support over at least a portion of the length of the treatment element. The at least one mounting tube comprises at least one abutment, said abutment being engagable with the support to limit axial movement of the treatment element. The support may comprise at least one abutment, said abutment being engagable with the at least one mounting tube to limit axial movement of the treatment element. Preferably the support abutment comprises a step on the support. In one variant the step on the support comprises a collar, a tube, a cir-clip, or a spring element mounted on the support and at least partially encircling the support. In another case the step on the support comprises a recess in the support.

In another embodiment the inside diameter of the at least one mounting tube is less than the outside diameter of the step. The at least one mounting tube may extend at least part of the length of the treatment element. In one case the at least one mounting tube extends distal of the treatment element. In another case the at least one mounting tube extends proximal of the treatment element.

The step on the support may comprise a collar and said collar comprises two abutment surfaces at either end of the collar. The collar engages with at least one mounting tube abutment surface and said engagement occurs within the body of the treatment element. In one embodiment the treatment element is substantially sealingly interfaced with the support. In another case the treatment element is substantially sealingly interfaced with the support along at least a portion of the length of the treatment element. The treatment element may be substantially sealingly interfaced with the support at the distal end and/or the proximal end of the treatment element.

Preferably the treatment element is moveable axially relative to the support. In another case the treatment element is moveable rotationally relative to the support. Where the cross sectional shape of the treatment element is not cylindrical this allows the treatment element to orient its major axis with the line of coaptation.

Preferably the mounting tube is isolated from blood contact. Preferably the support abutment surface is isolated from blood contact. Clot tends to form on stepped surfaces in vivo so these features are especially important.

In one embodiment the collar comprises an abutment surface at one end and a transition surface at the other end and said abutment surface engages with at least one mounting tube abutment surface. The transitioned surface may be blood contacting.

Preferably the collar is at least partially moveable along the support by the user. In one case the moveable collar is moved to frictionally engage with the mounting tube abutment surface so as to limit the rotational movement of the treatment element. In another embodiment the mounting tube comprises an inner diameter and at least one abutment surface and said mounting tube is formed in the treatment element. In another embodiment the treatment element comprises a reinforcement element.

In another embodiment the device comprises a delivery device, the delivery device comprising a distal end, a proximal end, and configured to advance the treatment element and deploy the treatment element at the treatment location. With this embodiment the delivery device comprises a slack promoting region.

The slack promoting region of the delivery device comprises a region wherein the delivery device has a low mechanical resistance to changes in its radius of curvature in response to compressive or tensile forces. Preferably the slack promoting region of the delivery device in use comprises a curved segment and the shape of said curved segment changes during the cardiac cycle.

In a variant of this embodiment the delivery device comprises a delivery catheter with a reception space at the distal end to house the treatment element in a collapsed state for delivery. Preferably the slack promoting region is proximal of the reception space. The delivery device may also comprise a support the support extending from the anchor to the treatment element. The treatment element is supported at the region of coaptation by the support. Preferably the support comprises a slack promoting region proximal of the anchor. In another case the support comprises a slack promoting region proximal of the treatment element.

In one case the slack promoting region of the delivery device comprises an extruded tubing of an elastomeric polymeric material. The elastomeric polymer may comprise a polymer with a Shore A hardness of less than 90. Preferably the elastomeric polymer comprises a polymer with a Shore A hardness of less than 80. Preferably the elastomeric polymer comprises a polymer with a Shore A hardness of less than 70. Preferably the elastomeric polymer comprises a polymer with a Shore A hardness of less than 60. The elastomeric polymer comprises a polymer with a Shore D hardness of less than 60. Preferably the elastomeric polymer comprises a polymer with a Shore D hardness of less than 55. Preferably the elastomeric polymer comprises a polymer with a Shore D hardness of less than 40.

In one embodiment the slack promoting region of the delivery device comprises a spring element with a soft outer jacket. Preferably the soft outer jacket comprises a polymer. In another construction the slack promoting region of the delivery device comprises a wire. The slack promoting region of the delivery device may comprise a flat wire. The slack promoting region of the delivery device may comprise at least one zone of articulation. In another embodiment the delivery device comprises a support and a catheter. Preferably both the support and the catheter comprise slack promoting segments.

This section 3 describes aspects of delivery devices and methods associated with the treatment elements. Additional features and embodiments are described in more detail in Section 4 of this statement and are incorporated in their entirety in this section 3 by reference.

In another embodiment the treatment element further comprises a reinforcement and an expandable body wherein the reinforcement at least partially restrains the expandable body at the region of coaptation. The treatment element comprise a collapsed delivery configuration and an expanded treatment configuration. With this embodiment expansion of the expandable body is triggered upon deployment of the expandable body in bodily fluid. The reinforcement may comprise a reinforcement layer which substantially encircles the expandable body in both the collapsed delivery configuration and in the expanded configuration. In one case the reinforcement comprises a braid, a mesh, a porous covering, a non-porous covering. Preferably the reinforcement layer comprises a biocompatible polymer.

Preferably the expandable body defines a small volume in the delivery configuration and a substantially larger volume in the expanded configuration. The increased volume defined by the expandable body in the expanded state is occupied by fluid inflow into the expandable body. In one case the expandable body comprises a hydrogel and the fluid inflow is retained in use by the expandable body. In one case the device further comprises a support, the support being connected to the treatment element and the support comprising an anchor at its distal end. In one embodiment the reinforcement layer is restrainedly connected to the support. In one case the reinforcement layer comprises a neck. The neck comprises a lumen and the support extends through said lumen. The neck is preferably mounted on the support. In one variant the neck is fixed to the support. The reinforcement may comprise a monofilament fibre. The reinforcement may comprise a knitted, woven or braided structure.

In another embodiment the device further comprises an anchor at an end of the support, the anchor configured to be engaged with a tissue wall. The device further comprises an anchor wire, the anchor wire is configured to transmit axial and/or torsional movements from a proximal end to the anchor, said axial and/or torsional movements facilitating the anchoring of the anchor to a wall of body tissue. The anchor wire comprises a coupling for transmission of said axial and torsional movements to the anchor.

The anchor wire comprises a proximal end and a distal end. The proximal end of the anchor wire extends proximal of the coupling. The distal end of the anchor wire extends distal of the coupling. In one embodiment the coupling comprises a pair of coupling features. The pair of coupling features comprises a proximal coupling feature and a distal coupling feature. The proximal coupling feature is preferably fixed to a proximal segment of the anchor wire and said distal coupling feature is preferably fixed to a distal segment of the anchor wire.

In one embodiment the proximal end of the anchor wire can be separated from the distal end of the anchor wire by decoupling the proximal coupling feature from the distal coupling feature. In one case the pair of coupling features comprises a male element and a female element. The pair of coupling features may comprise a taper lock coupling, a nut and bolt coupling, a flange coupling, a screw driver type coupling, a hex key type coupling, a snap fit coupling, a magnetic coupling, and a hook and ring type coupling. Preferably the pair of coupling features facilitate decoupling of the proximal and distal ends of the anchor wire. With this embodiment the anchor wire has a coupled configuration and a decoupled configuration. In the coupled configuration the anchor wire is configured to anchor the anchor to a body tissue wall. In the decoupled configuration the proximal end of the anchor wire is removable from the patient.

This section 3 describes aspects of anchoring the treatment elements. Additional features and embodiments are described in more detail in Section 6 of this statement and are incorporated in their entirety in this section 3 by reference.

In another embodiment the device further comprises an abutment stop configured to limit movement of the treatment element relative to the anchor. The abutment stop comprises an engagement surface. The abutment stop is connected to the anchor. The abutment stop may be integral with the support element.

In one embodiment the treatment element further comprises at least one engagement surface and engagement of the at least one treatment element engagement surface with the at least one abutment stop limits the axial movement of the treatment element along the support element. The at least one treatment element engagement surface may comprise at least one collar and said at least one collar engages with said at least one abutment stop to limits the axial movement of the treatment element along the support element. In use the abutment stop is implanted in the patient but is preferably shielded from direct contact with flowing blood. In one case the abutment stop is shielded from direct contact with flowing blood by positioned it within the body of the treatment element. The abutment stop may be positioned between the distal end and the proximal end of the treatment element. In this case the engagement between said abutment stop and said engagement surface may occur between the distal and proximal end of the treatment element. Preferably the support element abutment stop comprises a step on the outer surface of the support. The step on the outer surface of the support comprises a collar. Preferably the collar comprises at least one abutment surface. The support element abutment stop comprises a step on the inner surface of the support. The step on the inner surface of the support may comprise a recess.

In one embodiment the abutment stop is mounted to a tether. The tether comprises a flexible cable. Preferably the tether is strong in tensile. The tether may be soft in compression. With this embodiment the support comprises an inner lumen and said tether extends through at least a portion of said lumen. The tether limits movement of the treatment element away from the anchor. In one case the support limits movement of the treatment element towards the anchor. Preferably the anchor element is located at the distal end of the support element. In another embodiment the anchor is located proximal of the distal end of the support element.

In another embodiment the anchor penetrates the tissue wall in use. The device further comprises an anchor limiter. The penetration of the tissue wall by the anchor element is limited by an anchor limiter. The anchor limiter preferably engages a wall of tissue at the site of anchoring. In one case the anchor limiter comprises a tissue engagement element. The anchor limiter may engage the surface of a tissue wall at the site of anchoring. In one embodiment the anchor limiter at least partially penetrates the surface of the tissue wall at the site of anchoring. Preferably the anchor limiter is adjacent the anchor.

In one case the anchor limiter is expandable. With this embodiment the anchor limiter has an expanded configuration and a collapsed configuration. In the expanded configuration the anchor limiter comprises at least one arm. Preferably the at least one arm comprises a strut. In one embodiment the anchor limiter is deployed from a delivery device. The material of the anchor limiter may comprise a polymer, a metal, a stainless steel, a nitinol, a shape memory material, a super elastic material, a stainless alloy. Preferably the anchor limiter is a nitinol element with an expanded state and a collapsed state. Preferably the anchor limiter comprises arms extending radially outwardly in the expanded state. The anchor limiter may be manufactured from a hypotube. Preferably the anchor limiter comprises a slotted hypotube.

In one embodiment the anchor limiter interacts with the tissue wall surface to reinforce the grip of the anchor. Preferably the anchor limiter interacts with the tissue wall and prevents the anchor from disengaging with the tissue wall. The anchor limiter may prevent the anchor from unscrewing from the tissue wall. In one embodiment the anchor limiter comprises a cuff. The cuff comprises an engagement surface.

### Section 4

According to the invention there is provided a medical device suitable for use in treatment of a valve, the device comprising a treatment element, an anchor element, and a delivery device wherein, the treatment element is configured to be located at the region of coaptation of leaflets of a valve to resist fluid flow in a retrograde direction through an opening of the valve, the anchor element is configured to be anchored to a heart wall and said heart wall is in cyclical motion relative to said valve, the delivery device comprising a distal end, a proximal end, and configured to advance the treatment element and deploy the treatment element at the treatment location, wherein the delivery device comprises a slack promoting region.

In one embodiment the slack promoting region of the delivery device comprises a region wherein the delivery device has a low mechanical resistance to changes in its radius of curvature in response to compressive or tensile forces. The slack promoting region of the delivery device in use may comprise a curved segment and the shape of said curved segment may change during the cardiac cycle. In one variant the delivery device comprises a delivery catheter with a reception space at the distal end to house the treatment element in a collapsed state for delivery. Preferably the slack promoting region is proximal of the reception space.

In another embodiment the delivery device comprises a support the support extending from the anchor to the treatment element. The treatment element is supported at the region of coaptation by the support. In one case the support comprises a slack promoting region proximal of the anchor. In another case the support comprises a slack promoting region proximal of the treatment element. The slack promoting region of the delivery device may comprise an extruded tubing of an elastomeric polymeric material. Preferably the elastomeric polymer comprises a polymer with a Shore A hardness of less than 90. Preferably the elastomeric polymer comprises a polymer with a Shore A hardness of less than 80. Preferably the elastomeric polymer comprises a polymer with a Shore A hardness of less than 70. Preferably the elastomeric polymer comprises a polymer with a Shore A hardness of less than 60. Preferably the elastomeric polymer comprises a polymer with a Shore D hardness of less than 60. Preferably the elastomeric polymer comprises a polymer with a Shore D hardness of less than 50. Preferably the elastomeric polymer comprises a polymer with a Shore D hardness of less than 40.

In one embodiment the slack promoting region of the delivery device comprises a spring element with a soft outer jacket. The soft outer jacket comprises a polymer. In another embodiment the slack promoting region of the delivery device comprises a wire. The slack promoting region of the delivery device may comprise a flat wire. In one case the slack promoting region of the delivery device comprises at least one zone of articulation.

In another embodiment the delivery device comprises a support element and a catheter. Both the support element and the catheter comprise slack promoting segments. Preferably the slack promoting segments of the support element and the catheter are adjacent. In one variant the catheter comprises a reception space at its distal end. The treatment element is housed in said reception space during delivery.

In another case the delivery device comprises an advancement element and said advancement element extends from the treatment element to a proximal end to facilitate positioning of the treatment element at the region of coaptation. The advancement element also facilitates deployment of the treatment element at the region of coaptation. In another embodiment the advancement element facilitates expansion of the treatment element at the region of coaptation.

In one embodiment the delivery device comprises an anchor wire.

In yet another embodiment at least a portion of the delivery device is removable from the patient. Equally, at least a portion of said delivery device is implantable in the patient. In one case the implantable portion of said delivery device comprises a slack promoting region.

In one embodiment the delivery device comprises an inner shaft and an outer catheter and wherein at least the outer catheter is removable from the patient after deployment of the treatment element at the region of coaptation.

In another embodiment the delivery device comprises an inner shaft and an outer catheter and wherein the inner shaft is removable from the patient after deployment of the treatment element at the region of coaptation.

In another embodiment the slack promoting region comprises a region of the delivery device wherein the bending stiffness of the delivery device is low relative to adjacent regions of the delivery device. In one case the bending stiffness of the delivery device proximal of the treatment element is less than the bending stiffness distal of the delivery device. In another case the delivery device comprises a distal segment and said distal segment is constructed such that at least a substantial portion of the delivery device comprises a slack promoting segment.

In another embodiment the device further comprises at least one mounting tube. The support is configured to anchor the treatment element to a tissue wall, the treatment element being mounted to at least one mounting tube. The at least one mounting tube is rotatable relative to the support. The support extends from the tissue wall and supports the treatment element at the region of coaptation.

Preferably the treatment element is sealingly mounted to the at least one mounting tube. The at least one mounting tube may be integral with the treatment element. The mounting tube may be an extruded tube. The material of the mounting tube is preferably a polymer, or a metal. Preferably the material of the mounting is a biocompatible polymer or metal.

In one embodiment the mounting of the treatment element on the mounting tube comprises an interface layer. The interface layer comprises a mixture of mounting tube material and treatment element material. The interface layer is preferably at least partially resistant to fluid flow. The interface layer may be formed in a process that involves the local flow of polymer of at least one of the treatment element or the mounting tube. The local flow of polymer may created in a welding process or a solvent bonding process.

In one embodiment the support extends through the treatment element and the at least one mounting tube encircles the support over at least a portion of the length of the treatment element. The at least one mounting tube comprises at least one abutment, said abutment is engagable with the support to limit axial movement of the treatment element. In one case the support comprises at least one abutment, said abutment being engagable with the at least one mounting tube to limit axial movement of the treatment element.

In one embodiment the support abutment comprises a step on the support. The step on the support may comprises a collar, a tube, a cir-clip, or a spring element mounted on the support and at least partially encircling the support. In one case the step on the support comprises a recess in the support.

The inside diameter of the at least one mounting tube is less than the outside diameter of the step. In one embodiment the at least one mounting tube extends at least part of the length of the treatment element. In another embodiment the at least one mounting tube extends distal of the treatment element. In yet another embodiment the at least one mounting tube extends proximal of the treatment element.

The step may comprise a collar and said collar comprises two abutment surfaces at either end of the collar. The collar may engage with at least one mounting tube abutment surface and preferably said engagement occurs within the body of the treatment element. Preferably the treatment element is substantially sealingly interfaced with the support. The treatment element is preferably substantially sealingly interfaced with the support along at least a portion of the length of the treatment element. The treatment element may be substantially sealingly interfaced with the support at the distal end and/or the proximal end of the treatment element.

In one embodiment the treatment element is moveable axially relative to the support. In another case the treatment element is moveable rotationally relative to the support. Preferably the mounting tube is isolated from blood contact. Preferably the support abutment surface is isolated from blood contact. In one embodiment the collar comprises an abutment surface at one end and a transition surface at the other end and said abutment surface may engage with at least one mounting tube abutment surface. In one case the at least one abutment surface comprises a collar and said collar is at least partially moveable along the support by the user. The moveable collar is moveable to frictionally engage with the mounting tube abutment surface so as to limit the rotational movement of the treatment element. The mounting tube comprises an inner diameter and at least one abutment surface and said mounting tube is formed in the treatment element. In one embodiment the treatment element comprises a reinforcement element.

In another embodiment the treatment element further comprising a reinforcement and an expandable body wherein the reinforcement at least partially restrains the expandable body at the region of coaptation. The treatment element comprises a collapsed delivery configuration and an expanded treatment configuration. Expansion of the expandable body is triggered upon deployment of the expandable body in bodily fluid.

In one embodiment the reinforcement comprises a reinforcement layer which substantially encircles the expandable body in both the collapsed delivery configuration and in the expanded configuration. The reinforcement may comprise a braid, a mesh, a porous covering, a non-porous covering. Preferably the reinforcement layer further comprises a biocompatible polymer. The expandable body defines a small volume in the delivery configuration and a substantially larger volume in the expanded configuration. The increased volume defined by the expandable body in the expanded state is occupied by fluid inflow into the expandable body.

In one embodiment the expandable body comprises a hydrogel and the fluid inflow is retained in use by the expandable body. The device may further comprise a support, the support extending between the anchor and the treatment element, the support supporting the treatment element at the region of coaptation. In one case the reinforcement layer is restrainedly connected to the support. The reinforcement layer may comprise a neck. The neck may comprise a lumen and the support may extend through said lumen. The neck may be mounted on the support. The neck may be fixed to the support. In one case the reinforcement comprises a monofilament fibre. The reinforcement may comprise a knitted, woven or braided structure.

In another embodiment the device further comprises an anchor wire. The anchor wire is configured to transmit axial and/or torsional movements from a proximal end to the anchor, said axial and/or torsional movements facilitating the anchoring of the anchor to a wall of body tissue. The anchor wire comprises a coupling for transmission of said axial and torsional movements to the anchor. The anchor wire comprises a proximal end and a distal end. The proximal end of the anchor wire may extend proximal of the coupling. The distal end of the anchor wire may extend distal of the coupling.

In one embodiment the coupling comprises a pair of coupling features. The pair of coupling features may comprise a proximal coupling feature and a distal coupling feature. The proximal coupling feature may be fixed to a proximal segment of the anchor wire and said distal coupling feature may be fixed to a distal segment of the anchor wire.

In another embodiment the proximal end of the anchor wire can be separated from the distal end of the anchor wire by decoupling the proximal coupling feature from the distal coupling feature. In one case the pair of coupling features comprises a male element and a female element. The pair of coupling features may comprise a taper lock coupling, a nut and bolt coupling, a flange coupling, a screw driver type coupling, a hex key type coupling, a snap fit coupling, a magnetic coupling, and a hook and ring type coupling.

In another embodiment the pair of coupling features facilitate decoupling of the proximal and distal ends of the anchor wire. Thus, the anchor wire has a coupled configuration and a decoupled configuration. In the coupled configuration the anchor wire is configured to anchor the anchor to a body tissue wall. In the decoupled configuration the proximal end of the anchor wire is removable from the patient.

In one embodiment the anchor wire comprises a high modulus material.

In another embodiment the anchor wire comprises an inner shaft and an outer shaft. With this embodiment the anchor wire is anchored to the wall of body tissue by relative movement of the inner shaft and the outer shaft. The relative movement of the inner shaft and the outer shaft causes the anchor to anchor to the tissue wall. The relative movement of the inner shaft and the outer shaft may cause the anchor to expand in the tissue wall. The relative movement of the inner shaft and the outer shaft may cause the anchor to be deployed in the tissue wall.

In another case at least one of the pair of coupling features comprises a shaped element. The shaped element may be formed from the anchor wire and be integral with the anchor wire. Preferably the diameter of at least one of the pair of coupling features is small relative to the diameter of the treatment element. Preferably the diameter of at least one of the pair of coupling features is less than three times the diameter of the anchor wire. More preferably the diameter of at least one of the pair of coupling features is less than two times the diameter of the anchor wire. More preferably the diameter of at least one of the pair of coupling features is less than or equal to the diameter of the anchor wire.

The distal coupling feature may comprise at least one smooth transitioned surface. The distal coupling feature may be shielded from direct contact with flowing blood when implanted. The distal coupling feature may be shielded from contact with flowing blood by positioning the coupling feature within the body of the treatment element. The distal coupling feature may be located proximal of the treatment element. The distal coupling feature may located distal of the treatment element.

In another embodiment the support comprises a lumen. The distal coupling feature may be located within the lumen of the support. The distal coupling feature may be located adjacent or within the anchor.

In one case the anchor element is located at the distal end of the anchor wire. The anchor element may comprise a support over at least a portion of its length. The treatment element is supported at the region of coaptation by the support.

In another embodiment the device further comprises an abutment stop configured to limit movement of the treatment element relative to the anchor. The abutment stop comprises an engagement surface. The abutment stop is preferably connected to the anchor. The abutment stop may be integral with the support element.

In one case the treatment element comprises at least one engagement surface and engagement of the at least one treatment element engagement surface with the at least one abutment stop limits the axial movement of the treatment element along the support element. The at least one treatment element engagement surface may comprise at least one collar and said at least one collar engages with said at least one abutment stop to limits the axial movement of the treatment element along the support element.

Preferably, in use the abutment stop is implanted in the patient but is shielded from direct contact with flowing blood. The abutment stop may be shielded from direct contact with flowing blood by positioned it within the body of the treatment element.

In one case the abutment stop may be positioned between the distal end and the proximal end of the treatment element. In another case the abutment stop may be positioned between the distal end and the proximal end of the treatment element. Engagement between said abutment stop and said engagement surface may occur between the distal and proximal end of the treatment element.

In one embodiment the support element abutment stop may comprise a step on the outer surface of the support. The step on the outer surface of the support may comprise a collar. Preferably the collar comprises at least one abutment surface.

In one construction the support element abutment stop comprises a step on the inner surface of the support. Preferably the step on the inner surface of the support comprises recess. In this case the abutment stop is be mounted to a tether. Preferably the tether comprises a flexible cable, and the tether is strong in tensile. The tether may be soft in compression. The support comprises an inner lumen and said tether extends through at least a portion of said lumen. The tether limits movement of the treatment element away from the anchor. The support limits movement of the treatment element towards the anchor. The anchor element is located at the distal end of the support element. The anchor element is located at the proximal end of the support element.

In another embodiment the anchor at least partially penetrates the tissue wall in use and the device further comprises an anchor limiter. The penetration of the anchor element into the tissue wall is limited by an anchor limiter. The anchor limiter engages a wall of tissue at the site of anchoring. The anchor limiter comprises a tissue engagement element. The anchor limiter may engage the surface of a tissue wall at the site of anchoring.

In one case the anchor limiter at least partially penetrates the surface of the tissue wall at the site of anchoring. Preferably the anchor limiter is adjacent the anchor.

In one embodiment the anchor limiter is expandable. Thus, the anchor limiter has an expanded configuration and a collapsed configuration. In the expanded configuration the anchor limiter may comprise at least one arm. The at least one arm may comprises a strut. In one case the anchor limiter may be deployed from a delivery device. In another case the anchor limiter is activated by the anchor wire.

In another embodiment the material of the anchor limiter comprises a polymer, a metal, a stainless steel, a nitinol, a shape memory material, a super elastic material, a stainless alloy. Preferably the anchor limiter is a nitinol element with an expanded state and a collapsed state. The anchor limiter comprises arms extending radially outwardly in the expanded state. The anchor limiter may be manufactured from a hypotube. In one case the anchor limiter comprises a slotted hypotube.

In one case the anchor limiter interacts with the tissue wall surface to reinforce the grip of the anchor. The anchor limiter may interact with the tissue wall and so prevent the anchor from disengaging with the tissue wall. In one embodiment the anchor limiter prevents the anchor from unscrewing from the tissue wall. In another embodiment the anchor limiter comprises a cuff. The cuff comprises an engagement surface.

In another embodiment the treatment element comprises a hydrogel. The treatment element further comprises a support for supporting the treatment element at the region of coaptation, the support being connected to the anchor element. The treatment element is anchored to a heart wall comprises a ventricle wall, an atrial wall, or a septal wall.

The treatment element has a dehydrated state and a hydrated state and the dehydrated state corresponds to the collapsed delivery configuration and the hydrated state corresponds to the expanded treatment configuration. In the hydrated state the hydrogel comprises primarily of polymer and a hydrating liquid and at least some of the polymer molecules are cross-linked.

The hydrating liquid may comprise saline, contrast media, a biocompatible fluid, silicone fluid, or blood plasma components. In the expanded hydrated state the hydrogel is mostly liquid with solid polymer network spanning the occupied volume. Preferably the hydrogel comprises at least 50% liquid by volume in the hydrated state. More preferably the hydrogel comprises at least 70% liquid by volume in the expanded hydrated state. Yet more preferably the hydrogel comprises at least 80% liquid by volume in the expanded hydrated state. More preferably the hydrogel comprises at least 90% liquid by volume in the expanded hydrated state. More preferably the hydrogel comprises at least 95% liquid by volume in the expanded hydrated state. Even more preferably the hydrogel comprises from 95% to 99% liquid by volume in the expanded hydrated state.

In one embodiment the density of the treatment element is in the range 1200 kg/m³ to 1025 kg/m³ in the expanded state. The polymer network of the hydrogel is at least partially composed of a synthetic polymer, a protein or a natural polymer.

Preferably the compliance of the treatment element in the expanded hydrated state is greater than the compliance of the treatment element in the collapsed state. The polymer network of the hydrogel may be loaded with an active compound which is eluted from the hydrogel over time. The eluted compound is selected from one or more of an anticoagulant, an anti-thrombin, an anti-platelet, an agent to prevent thrombosis, an anti-proliferative, an anti-fibrotic, an agent to promote endothelialisation, and a drug. The compound comprises heparin or a factor Xa inhibitor.

In one embodiment the hydrogel is porous. In another case the hydrogel is at least partially solid.

The hydrogel comprises hydrophilic chain segments. Preferably the hydrophilic chain segments comprise high electronegativity atoms and said high electronegativity atoms comprise at least 10% of the atomic mass of the chain segment. More preferably the hydrophilic chain segments comprise high electronegativity atoms and said high electronegativity atoms comprise at least 20% of the atomic mass of the chain segment. More preferably the hydrophilic chain segments comprise high electronegativity atoms and said high electronegativity atoms comprise at least 25% of the atomic mass of the chain segment. Most preferably the hydrophilic chain segments comprise high electronegativity atoms and said high electronegativity atoms comprise at least 30% of the atomic mass of the chain segment.

In one embodiment the polymer of the hydrogel is based on one or more of polyvinyl alcohol (PVA), sodium polyacrylate, hydrophilic acrylate polymers, hydrophilic polymethacrylates, 2-hydroxyethyl-methacrylate (HEMA), ethylene glycol bismethacrylate, hyluronan polymers, poly(anhydride esters), poly(vinylpyrroldine), poly(ethyloxazoline), poly(ethylene glycol)-co-poly(propylene glycol) block copolymers, hydrophilic methacrylamides, and a polyethylene glycol based polyurethane.

In another embodiment the treatment element is sonolucent. The sonolucent treatment element can be contrasted with surrounding tissue and can be imaged using echocardiograph. In one case the hydrating liquid at least comprises a contrast medium.

### Section 5

According to the invention there is provided a medical device suitable for use in treatment of a valve, the valve being movable between a closed configuration and an open configuration, the device comprising a treatment element configured to be located at the region of co-aptation of leaflets of a valve to resist fluid flow in a retrograde direction through an opening of the valve, the treatment element comprising a collapsed delivery configuration and an expanded treatment configuration, the treatment element comprising a reinforcement and an expandable body wherein the reinforcement at least partially restrains the expandable body at the region of coaptation.

Expansion of the expandable body is activated upon deployment of the expandable body in bodily fluid.

The reinforcement comprises a reinforcement layer which substantially encircles the expandable body in both the collapsed delivery configuration and in the expanded configuration. The reinforcement comprises a braid, a mesh, a porous covering, a non-porous covering. The reinforcement layer may comprise a biocompatible polymer. The expandable body defines a small volume in the delivery configuration and a substantially larger volume in the expanded configuration. The increased volume defined by the expandable body in the expanded state is occupied by fluid inflow into the expandable body. In one case the expandable body comprises a hydrogel and the fluid inflow is retained in use by the expandable body.

In one case the device comprises a support element, the support element being connected to the treatment element and the support element comprising an anchor at its distal end. The reinforcement layer is restrainedly connected to the support element. The reinforcement layer may comprise a neck. The neck comprises a lumen and the support extends through said lumen. The neck may be mounted on the support element. The neck may be fixed to the support element. The reinforcement may comprise a monofilament fibre. The reinforcement may comprise a knitted, woven or braided structure.

In another embodiment the anchor element is configured to be anchored to a heart wall and said heart wall is in cyclical motion relative to said valve. The device further comprises a delivery device, the delivery device comprising a distal end, a proximal end, and configured to advance the treatment element and deploy the treatment element at the treatment location. The delivery device comprises a slack promoting region.

In one case the slack promoting region of the delivery device comprises a region wherein the delivery device has a low mechanical resistance to changes in its radius of curvature in response to compressive or tensile forces. The slack promoting region of the delivery device in use comprises a curved segment and the shape of said curved segment changes during the cardiac cycle.

In one embodiment the delivery device comprises a delivery catheter with a reception space at the distal end to house the treatment element in a collapsed state for delivery. The slack promoting region is preferably proximal of the reception space. In one case the delivery device comprises a support the support extending from the anchor to the treatment element. The treatment element is supported at the region of coaptation by the support. The support may comprise a slack promoting region proximal of the anchor. The support comprises a slack promoting region proximal of the treatment element.

In one case the slack promoting region of the delivery device comprises an extruded tubing of an elastomeric polymeric material. The elastomeric polymer comprises a polymer with a Shore A hardness of less than 90. Preferably the elastomeric polymer comprises a polymer with a Shore A hardness of less than 80. Preferably the elastomeric polymer comprises a polymer with a Shore A hardness of less than 70. Preferably the elastomeric polymer comprises a polymer with a Shore A hardness of less than 60. The elastomeric polymer comprises a polymer with a Shore D hardness of less than 60. The elastomeric polymer comprises a polymer with a Shore D hardness of less than 50. The elastomeric polymer comprises a polymer with a Shore D hardness of less than 40.

In another embodiment the slack promoting region of the delivery device comprises a spring element with a soft outer jacket. The soft outer jacket comprises a polymer. Alternatively, the slack promoting region of the delivery device comprises a wire. The slack promoting region of the delivery device may comprise a flat wire. The slack promoting region of the delivery device may comprise at least one zone of articulation.

In another embodiment the delivery device comprises a support element and a catheter. Both the support element and the catheter comprise slack promoting segments. In one case the slack promoting segments of the support element and the catheter are adjacent. In another embodiment the catheter comprises a reception space at its distal end. The treatment element is housed in said reception space during delivery.

In another case the delivery device comprises an advancement element and said advancement element extends from the treatment element to a proximal end to facilitate positioning of the treatment element at the region of coaptation. The advancement element facilitates deployment of the treatment element at the region of coaptation. The advancement element facilitates expansion of the treatment element at the region of coaptation.

In another embodiment the delivery device comprises an anchor wire.

In another embodiment at least a portion of said delivery device is removable from the patient. Conversely, at least a portion of said delivery device is implantable in the patient. The implantable portion of said delivery device may also comprise a slack promoting region.

In yet another embodiment the delivery device comprises an inner shaft and an outer catheter and wherein at least the outer catheter is removable from the patient after deployment of the treatment element at the region of coaptation.

In yet another case the delivery device comprises an inner shaft and an outer catheter and wherein the inner shaft is removable from the patient after deployment of the treatment element at the region of coaptation.

The slack promoting region comprises a region of the delivery device wherein the bending stiffness of the delivery device is low relative to adjacent regions of the delivery device. In one case the bending stiffness of the delivery device proximal of the treatment element is less than the bending stiffness distal of the delivery device. In another the delivery device comprises a distal segment and said distal segment is constructed such that at least a substantial portion of the delivery device comprises a slack promoting segment.

In another embodiment the device further comprises at least one mounting tube. The support is configured to anchor the treatment element to a tissue wall, the treatment element being mounted to at least one mounting tube. The at least one mounting tube is rotatable relative to the support. The support extends from the tissue wall and supports the treatment element at the region of coaptation.

In one variant the treatment element is sealingly mounted to the at least one mounting tube. The at least one mounting tube may be integral with the treatment element. The mounting tube may comprise an extruded tube. The material of the mounting may be a polymer, or a metal. The material of the mounting is preferably a biocompatible polymer or metal. The material of the mounting tube comprises a polyurethane, a polyether polyurethane, a polycarbonate polyurethane, a polydimethysiloxane polyurethane, a silicone, a fluoropolymer, a polyester, polyethylene terephthalate, polyethylene naphthalate, a polyolefin, a polyethylene, ultra high molecular weight polyethylene, polyetheretherketone (PEEK), polyether ketone (PEK), stainless steel, a stainless alloy, a super elastic metal, a shape memory metal, and a nitinol.

In one embodiment the mounting of the treatment element on the mounting tube comprises an interface layer. The interface layer comprises a mixture of mounting tube material and treatment element material. The interface layer is preferably at least partially resistant to fluid flow. The interface layer may be formed in a process that involves the local flow of polymer of at least one of the treatment element or the mounting tube. The local flow of polymer may be created in a welding process or a solvent bonding process.

In one case the support extends through the treatment element and the at least one mounting tube encircles the support over at least a portion of the length of the treatment element. In one case the at least one mounting tube comprises at least one abutment, said abutment being engagable with the support to limit axial movement of the treatment element. In another embodiment the support comprises at least one abutment, said abutment being engagable with the at least one mounting tube to limit axial movement of the treatment element. The support abutment may comprise a step on the support. The step on the support may comprise a collar, a tube, a cir-clip, or a spring element mounted on the support and at least partially encircling the support.

In one embodiment the step on the support comprises a recess in the support. The inside diameter of the at least one mounting tube is less than the outside diameter of the step. In one case the at least one mounting tube extends at least part of the length of the treatment element. In another case the at least one mounting tube extends distal of the treatment element. In another the at least one mounting tube extends proximal of the treatment element.

In one case the step comprises a collar and said collar comprises two abutment surfaces at either end of the collar. The collar engages with at least one mounting tube abutment surface and said engagement occurs within the body of the treatment element. The treatment element may be substantially sealingly interfaced with the support. The treatment element may be substantially sealingly interfaced with the support along at least a portion of the length of the treatment element. The treatment element may be substantially sealingly interfaced with the support at the distal end and/or the proximal end of the treatment element.

In one embodiment the treatment element is moveable axially relative to the support. In another the treatment element is moveable rotationally relative to the support. Preferably the mounting tube is isolated from blood contact. Preferably the support abutment surface is isolated from blood contact.

In one case the collar comprises an abutment surface at one end and a transition surface at the other end and said abutment surface engages with at least one mounting tube abutment surface. The at least one abutment surface comprises a collar and said collar is at least partially moveable along the support by the user. The moveable collar is moveable to frictionally engage with the mounting tube abutment surface so as to dampen the rotational movement of the treatment element. The mounting tube comprises an inner diameter and at least one abutment surface and said mounting tube is formed in the treatment element.

In an other embodiment the device further comprises an anchor and an anchor wire, wherein the anchor is configured to anchor the treatment element to a tissue wall. The anchor wire is configured to transmit axial and/or torsional movements from a proximal end to the anchor, said axial and/or torsional movements facilitating the anchoring of the anchor to a wall of body tissue. The anchor wire comprises a coupling for transmission of said axial and torsional movements to the anchor. The anchor wire further comprises a proximal end and a distal end.

The proximal end of the anchor wire extends proximal of the coupling. The distal end of the anchor wire extends distal of the coupling. Preferably the coupling comprises a pair of coupling features. The pair of coupling features may comprise a proximal coupling feature and a distal coupling feature. The proximal coupling feature may be fixed to a proximal segment of the anchor wire and said distal coupling feature may be fixed to a distal segment of the anchor wire.

In one embodiment the proximal end of the anchor wire can be separated from the distal end of the anchor wire by decoupling the proximal coupling feature from the distal coupling feature.

In one embodiment the pair of coupling features comprises a male element and a female element. The pair of coupling features may comprise a taper lock coupling, a nut and bolt coupling, a flange coupling, a screw driver type coupling, a hex key type coupling, a snap fit coupling, a magnetic coupling, and a hook and ring type coupling. The pair of coupling features facilitates decoupling of the proximal and distal ends of the anchor wire. Thus the anchor wire has a coupled configuration and a decoupled configuration. In the coupled configuration the anchor wire is configured to anchor the anchor to a body tissue wall. In the decoupled configuration the proximal end of the anchor wire is removable from the patient.

In another case the anchor wire comprises a high modulus material. In another embodiment the anchor wire comprises an inner shaft and an outer shaft. The anchor wire may be anchored to the wall of body tissue by relative movement of the inner shaft and the outer shaft. The relative movement of the inner shaft and the outer shaft causes the anchor to anchor to the tissue wall. In one variant the relative movement of the inner shaft and the outer shaft causes the anchor to expand in the tissue wall. In another the relative movement of the inner shaft and the outer shaft causes the anchor to be deployed in the tissue wall.

In one case at least one of said pair of coupling features comprise a shaped element. The shaped element is formed from the anchor wire and is integral with the anchor wire. Preferably the diameter of at least one of the pair of coupling features is small relative to the diameter of the treatment element. More preferably the diameter of at least one of the pair of coupling features is less than three times the diameter of the anchor wire. More preferably the diameter of at least one of the pair of coupling features is less than two times the diameter of the anchor wire. Most preferably the diameter of at least one of the pair of coupling features is less than or equal to the diameter of the anchor wire.

The distal coupling feature comprises at least one smooth transitioned surface. The distal coupling feature is preferably shielded from direct contact with flowing blood when implanted. The distal coupling feature is preferably shielded from contact with flowing blood by positioning the coupling feature within the body of the treatment element.

In one case the distal coupling feature is located proximal of the treatment element. The distal coupling feature is located distal of the treatment element. In one variant the support further comprises a lumen and the distal coupling feature is located within a lumen of the support. In another case the distal coupling feature is located adjacent or within the anchor. The anchor element is located at the distal end of the anchor wire. The anchor element may comprise a support over at least a portion of its length. The treatment element is supported at the region of coaptation by the support.

In another embodiment the device further comprises an abutment stop configured to limit movement of the treatment element relative to the anchor. The abutment stop comprises an engagement surface. The abutment stop is connected to the anchor. The abutment stop is preferably integral with the support element.

In another embodiment the treatment element further comprises at least one engagement surface and engagement of the at least one treatment element engagement surface with the at least one abutment stop limits the axial movement of the treatment element along the support element. The at least one treatment element engagement surface comprises at least one collar and said at least one collar engages with said at least one abutment stop to limits the axial movement of the treatment element along the support element.

In use the abutment stop is implanted in the patient but is preferably shielded from direct contact with flowing blood. The abutment stop is preferably shielded from direct contact with flowing blood by positioned it within the body of the treatment element. The abutment stop may be positioned between the distal end and the proximal end of the treatment element. The abutment stop may be positioned between the distal end and the proximal end of the treatment element.

In one case engagement between said abutment stop and said engagement surface occurs between the distal and proximal end of the treatment element. The support element abutment stop may comprise a step on the outer surface of the support. The step on the outer surface of the support may comprise a collar. The collar comprises at least one abutment surface.

In another embodiment the support element abutment stop comprises a step on the inner surface of the support. The step on the inner surface of the support preferably comprises recess. With the recess feature the abutment stop can be mounted to a tether. The support comprises an inner lumen and said tether extends through at least a portion of said lumen. The tether limits movement of the treatment element away from the anchor. The tether comprises a flexible cable. The tether is strong in tensile. The tether may be soft in compression. The support limits movement of the treatment element towards the anchor.

The anchor element is preferably located at the distal end of the support element.

In another embodiment the anchor at least partially penetrates the tissue wall in use. The device further comprises an anchor limiter. The degree of penetration of the tissue wall by the anchor element is limited by an anchor limiter. The anchor limiter engages a wall of tissue at the site of anchoring. The anchor limiter comprises a tissue engagement element. The anchor limiter may engage the surface of a tissue wall at the site of anchoring. In one case the anchor limiter at least partially penetrates the surface of the tissue wall at the site of anchoring. The anchor limiter is preferably adjacent the anchor.

In one embodiment the anchor limiter is expandable. Thus the anchor limiter has an expanded configuration and a collapsed configuration. In the expanded configuration the anchor limiter comprises at least one arm. The at least one arm comprises a strut. In one case the anchor limiter is deployed from a delivery device.

In one case the material of the anchor limiter comprises a polymer, a metal, a stainless steel, a nitinol, a shape memory material, a super elastic material, a stainless alloy. Preferably the anchor limiter is a nitinol element with an expanded state and a collapsed state. The anchor limiter comprises arms extending radially outwardly in the expanded state. The anchor limiter may be manufactured from a hypotube. In one case the anchor limiter comprises a slotted hypotube.

In one case the anchor limiter interacts with the tissue wall surface to reinforce the grip of the anchor. The anchor limiter may interact with the tissue wall and prevents the anchor from disengaging with the tissue wall. The anchor limiter may prevent the anchor from unscrewing from the tissue wall. The anchor limiter comprises a cuff. The cuff comprises an engagement surface.

In another embodiment the treatment element comprises a hydrogel. The treatment element has a dehydrated state and a hydrated state and the dehydrated state corresponds to the collapsed delivery configuration and the hydrated state corresponds to the expanded treatment configuration. In the hydrated state the hydrogel comprises primarily of polymer and a hydrating liquid and at least some of the polymer molecules are cross-linked. In the hydrating liquid comprises saline, contrast media, a biocompatible fluid, silicone fluid, or blood plasma components. In the expanded hydrated state the hydrogel is mostly liquid with solid polymer network spanning the occupied volume. The hydrogel comprises at least 50% liquid by volume in the hydrated state. The hydrogel comprises at least 70% liquid by volume in the expanded hydrated state. The hydrogel comprises at least 80% liquid by volume in the expanded hydrated state. The hydrogel comprises at least 90% liquid by volume in the expanded hydrated state. The hydrogel comprises at least 95% liquid by volume in the expanded hydrated state. The hydrogel comprises from 95% to 99% liquid by volume in the expanded hydrated state.

In one embodiment the density of the treatment element is in the range 1200 kg/m³ to 1025 kg/m³ in the expanded state.

The polymer network of the hydrogel is at least partially composed of a synthetic polymer, a protein or a natural polymer. The compliance of the treatment element in the expanded hydrated state is greater than the compliance of the treatment element in the collapsed state.

The polymer network of the hydrogel may be loaded with an active compound which is eluted from the hydrogel over time. The eluted compound is selected from one or more of an anticoagulant, an anti-thrombin, an anti-platelet, an agent to prevent thrombosis, an anti-proliferative, an anti-fibrotic, an agent to promote endothelialisation, and a drug. The compound comprises heparin or a factor Xa inhibitor.

In one case the hydrogel is porous. In another the hydrogel is at least partially solid.

In one embodiment the hydrogel comprises hydrophilic chain segments. The hydrophilic chain segments comprise high electronegativity atoms and said high electronegativity atoms comprise at least 10% of the atomic mass of the chain segment. The hydrophilic chain segments comprise high electronegativity atoms and said high electronegativity atoms comprise at least 20% of the atomic mass of the chain segment. The hydrophilic chain segments comprise high electronegativity atoms and said high electronegativity atoms comprise at least 25% of the atomic mass of the chain segment. The hydrophilic chain segments comprise high electronegativity atoms and said high electronegativity atoms comprise at least 30% of the atomic mass of the chain segment.

In another embodiment the polymer of the hydrogel is based on one or more of polyvinyl alcohol (PVA), sodium polyacrylate, hydrophilic acrylate polymers, hydrophilic polymethacrylates, 2-hydroxyethyl-methacrylate (HEMA), ethylene glycol bismethacrylate, hyluronan polymers, poly(anhydride esters), poly(vinylpyrroldine), poly(ethyloxazoline), poly(ethylene glycol)-co-polypropylene glycol) block copolymers, hydrophilic methacrylamides, and a polyethylene glycol based polyurethane.

The treatment element is sonolucent. The hydrating liquid at least comprises a contrast medium.

### Section 6

According to the invention there is provided a medical device suitable for use in treatment of a valve, the device comprising a treatment element, an anchor element, and an anchor wire wherein,

the treatment element is configured to be located at the region of coaptation of leaflets of a valve to resist fluid flow in a retrograde direction through an opening of the valve, the anchor element is located at an end of the anchor wire and is configured to anchor the treatment element to a heart wall, the anchor wire extends from the anchor and supports the treatment element at the region of coaptation, wherein the anchor wire is configured to transmit axial and/or torsional movements from a proximal end to the anchor at the distal end, said axial and/or torsional movements facilitating the anchoring of the anchor to the heart wall, wherein, the anchor wire comprises a coupling for transmission of said axial and torsional movements to the anchor.

The anchor wire comprises a proximal end and a distal end. The proximal end of the anchor wire extends proximal of the coupling. The distal end of the anchor wire extends distal of the coupling.

In one case the coupling comprises a pair of coupling features. The pair of coupling features comprises a proximal coupling feature and a distal coupling feature. The proximal coupling feature is fixed to a proximal segment of the anchor wire and said distal coupling feature is fixed to a distal segment of the anchor wire. The proximal end of the anchor wire can be separated from the distal end of the anchor wire by decoupling the proximal coupling feature from the distal coupling feature.

In one embodiment the pair of coupling features comprises a male element and a female element.

In one embodiment the pair of coupling features comprise a taper lock coupling, a nut and bolt coupling, a flange coupling, a screw driver type coupling, a hex key type coupling, a snap fit coupling, a magnetic coupling, and a hook and ring type coupling.

In one case the pair of coupling features facilitates decoupling of the proximal and distal ends of the anchor wire. Thus the anchor wire has a coupled configuration and a decoupled configuration. In the coupled configuration the anchor wire is configured to anchor the anchor to a body tissue wall. In the decoupled configuration the proximal end of the anchor wire may be removable from the patient.

In one embodiment the anchor wire comprises a high modulus material. In another embodiment the anchor wire comprises an inner shaft and an outer shaft. The anchor wire is anchored to the wall of body tissue by relative movement of the inner shaft and the outer shaft. In one case the relative movement of the inner shaft and the outer shaft causes the anchor to anchor to the tissue wall. In another case the relative movement of the inner shaft and the outer shaft causes the anchor to expand in the tissue wall. In another the relative movement of the inner shaft and the outer shaft causes the anchor to be deployed in the tissue wall.

In one embodiment at least one of said pair of coupling features comprise a shaped element. The shaped element is formed from the anchor wire and is integral with the anchor wire. The diameter of at least one of the pair of coupling features is small relative to the diameter of the treatment element. Preferably the diameter of at least one of the pair of coupling features is less than three times the diameter of the anchor wire. More preferably the diameter of at least one of the pair of coupling features is less than two times the diameter of the anchor wire. Most preferably the diameter of at least one of the pair of coupling features is less than or equal to the diameter of the anchor wire.

In one embodiment the distal coupling feature comprises at least one smooth transitioned surface. The distal coupling feature may be shielded from direct contact with flowing blood when implanted. Preferably the distal coupling feature is shielded from contact with flowing blood by positioning the coupling feature within the body of the treatment element. The distal coupling feature may be located proximal of the treatment element. The distal coupling feature may be located distal of the treatment element.

In another embodiment the device further comprises a connector, to connect the treatment element and the anchor. The distal coupling feature may be located within a lumen of the connector element. The distal coupling feature may be located adjacent or within the anchor. The anchor element may be located at the distal end of the anchor wire. The anchor element may be located proximal of the distal end of the anchor wire.

In one embodiment the treatment element comprises a collapsed delivery configuration and an expanded treatment configuration, and the treatment element further comprising a reinforcement and an expandable body wherein the reinforcement at least partially restrains the expandable body at the region of coaptation.

In one case expansion of the expandable body is activated upon deployment of the expandable body in bodily fluid.

In another embodiment the reinforcement comprises a reinforcement layer which substantially encircles the expandable body in both the collapsed delivery configuration and in the expanded configuration. The reinforcement may comprise a braid, a mesh, a porous covering, a non-porous covering. The reinforcement layer may further comprises a biocompatible polymer. The expandable body defines a small volume in the delivery configuration and a substantially larger volume in the expanded configuration. The increased volume defined by the expandable body in the expanded state is occupied by fluid inflow into the expandable body.

In one case the expandable body comprises a hydrogel and the fluid inflow is retained in use by the expandable body.

In another case the device further comprises a support element, the support element being connected to the treatment element and the support element comprising an anchor at its distal end. The reinforcement layer is restrainedly connected to the support element. The reinforcement layer may comprise a neck. The neck may comprise a lumen and the support extends through said lumen. The neck may be mounted on the support element. The neck may be fixed to the support element.

In one case the reinforcement comprises a monofilament fibre. The reinforcement comprises a knitted, woven or braided structure.

In another embodiment the anchor element is configured to be anchored to a heart wall and said heart wall is in cyclical motion relative to said valve. The device further comprises a delivery device, the delivery device comprising a distal end, a proximal end, and configured to advance the treatment element and deploy the treatment element at the treatment location. The delivery device comprises a slack promoting region.

In one embodiment the slack promoting region of the delivery device comprises a region wherein the delivery device has a low mechanical resistance to changes in its radius of curvature in response to compressive or tensile forces. The slack promoting region of the delivery device in use may comprise a curved segment and the shape of said curved segment changes during the cardiac cycle.

In one embodiment the delivery device comprises a delivery catheter with a reception space at the distal end to house the treatment element in a collapsed state for delivery. The slack promoting region may be proximal of the reception space.

In another embodiment the delivery device comprises a support the support extending from the anchor to the treatment element. The treatment element is supported at the region of coaptation by the support. The support further comprises a slack promoting region proximal of the anchor. Alternatively the support comprises a slack promoting region proximal of the treatment element.

In one case the slack promoting region of the delivery device comprises an extruded tubing of an elastomeric polymeric material. The elastomeric polymer comprises a polymer with a Shore A hardness of less than 90. The elastomeric polymer comprises a polymer with a Shore A hardness of less than 80. The elastomeric polymer comprises a polymer with a Shore A hardness of less than 70. The elastomeric polymer comprises a polymer with a Shore A hardness of less than 60. The elastomeric polymer comprises a polymer with a Shore D hardness of less than 60. The elastomeric polymer comprises a polymer with a Shore D hardness of less than 50. The elastomeric polymer comprises a polymer with a Shore D hardness of less than 40.

In another embodiment the slack promoting region of the delivery device comprises a spring element with a soft outer jacket. The soft outer jacket comprises a polymer.

In another case the slack promoting region of the delivery device comprises a wire. In a variation of this the slack promoting region of the delivery device comprises a flat or flattened wire.

In another variant the slack promoting region of the delivery device comprises at least one zone of articulation.

In another embodiment the delivery device comprises a support and a catheter. Both the support and the catheter comprise slack promoting segments. The slack promoting segments of the support element and the catheter are preferably adjacent. The catheter may comprise a reception space at its distal end. The treatment element may be housed in said reception space during delivery.

In another case the delivery device comprises an advancement element and said advancement element extends from the treatment element to a proximal end to facilitate positioning of the treatment element at the region of coaptation. The advancement element may also facilitate deployment of the treatment element at the region of coaptation. The advancement element may be adapted to facilitate expansion of the treatment element at the region of coaptation.

In another case the delivery device comprises an anchor wire.

At least a portion of said delivery device is removable from the patient. At least a portion of said delivery device is implantable in the patient. In one case the implantable portion of said delivery device comprises a slack promoting region.

In another embodiment the delivery device comprises an inner shaft and an outer catheter and wherein at least the outer catheter is removable from the patient after deployment of the treatment element at the region of coaptation.

In another embodiment the delivery device comprises an inner shaft and an outer catheter and wherein the inner shaft is removable from the patient after deployment of the treatment element at the region of coaptation.

The slack promoting region comprises a region of the delivery device wherein the bending stiffness of the delivery device is low relative to adjacent regions of the delivery device. The bending stiffness of the delivery device proximal of the treatment element is less than the bending stiffness distal of the delivery device. The delivery device comprises a distal segment and said distal segment is constructed such that at least a substantial portion of the delivery device comprises a slack promoting segment.

In another embodiment the device further comprises at least one mounting tube. The treatment element is mounted to at least one mounting tube. The at least one mounting tube is rotatable relative to the support. The support extends from the tissue wall and supports the treatment element at the region of coaptation.

In one case the treatment element is sealingly mounted to the at least one mounting tube. The at least one mounting tube may be integral with the treatment element. In one case the mounting tube comprises an extruded tube. Preferably the material of the mounting is a polymer, or a metal. Preferably the material of the mounting is a biocompatible polymer or metal. The material of the mounting tube may comprise a polyurethane, a polyether polyurethane, a polycarbonate polyurethane, a polydimethysiloxane polyurethane, a silicone, a fluoropolymer, a polyester, polyethylene terephthalate, polyethylene naphthalate, a polyolefin, a polyethylene, ultra high molecular weight polyethylene, polyetheretherketone (PEEK), polyether ketone (PEK), stainless steel, a stainless alloy, a super elastic metal, a shape memory metal, a hydrogel and a nitinol.

In one case the mounting of the treatment element on the mounting tube comprises an interface layer. The interface layer comprises a mixture of mounting tube material and treatment element material. The interface layer is at least partially resistant to fluid flow. The interface layer is formed in a process that involves the local flow of polymer of at least one of the treatment element or the mounting tube. The local flow of polymer may be created in a welding process or a solvent bonding process.

In one embodiment the support extends through the treatment element and the at least one mounting tube encircles the support over at least a portion of the length of the treatment element. The at least one mounting tube comprises at least one abutment, said abutment being engagable with the support to limit axial movement of the treatment element.

The support comprises at least one abutment, said abutment being engagable with the at least one mounting tube to limit axial movement of the treatment element. In one case the support abutment comprises a step on the support. The step on the support may comprise a collar, a tube, a cir-clip, or a spring element mounted on the support and at least partially encircling the support.

The step on the support may comprise a recess in the support.

Preferably the inside diameter of the at least one mounting tube is less than the outside diameter of the step. In one case the at least one mounting tube extends at least part of the length of the treatment element. In another case the at least one mounting tube extends distal of the treatment element. In another case the at least one mounting tube extends proximal of the treatment element.

In one embodiment the step comprises a collar and said collar comprises two abutment surfaces at either end of the collar. The collar may engage with at least one mounting tube abutment surface and said engagement occurs within the body of the treatment element. The treatment element may be substantially sealingly interfaced with the support. The treatment element may be substantially sealingly interfaced with the support along at least a portion of the length of the treatment element. The treatment element may substantially sealingly interfaced with the support at the distal end and/or the proximal end of the treatment element.

In one embodiment the treatment element is moveable axially relative to the support. The treatment element may be moved rotationally relative to the support.

Preferably the mounting tube is isolated from blood contact. Preferably the support abutment surface is isolated from blood contact.

In another embodiment the collar comprises an abutment surface at one end and a transition surface at the other end and said abutment surface engages with at least one mounting tube abutment surface.

In another embodiment the at least one abutment surface comprises a collar and said collar is at least partially moveable along the support by the user. The moveable collar is moveable to frictionally engage with the mounting tube abutment surface so as to dampen the rotational movement of the treatment element.

In another case the mounting tube comprises an inner diameter and at least one abutment surface and said mounting tube is formed in the treatment element.

In another embodiment the device further comprises an abutment stop configured to limit movement of the treatment element relative to the anchor. The abutment stop comprises an engagement surface. The abutment stop is connected to the anchor. The abutment stop may be integral with the anchor wire.

In one embodiment the treatment element comprises at least one engagement surface and engagement of the at least one treatment element engagement surface with the at least one abutment stop limits the axial movement of the treatment element along the anchor wire. The at least one treatment element engagement surface comprises at least one collar and said at least one collar engages with said at least one abutment stop to limits the axial movement of the treatment element along the anchor wire.

In use the abutment stop is implanted in the patient but is preferably shielded from direct contact with flowing blood. The abutment stop may be shielded from direct contact with flowing blood by positioned it within the body of the treatment element. The abutment stop may be positioned between the distal end and the proximal end of the treatment element. The abutment stop may be positioned between the distal end and the proximal end of the treatment element. Engagement between said abutment stop and said engagement surface occurs between the distal and proximal end of the treatment element.

In one embodiment the anchor wire abutment stop comprises a step on the outer surface of the anchor wire. The step on the outer surface of the anchor wire may comprise a collar. The collar comprises at least one abutment surface.

In another embodiment the anchor wire abutment stop comprises a step on the inner surface of the anchor wire. The step on the inner surface of the anchor wire may comprise a recess. The abutment stop may be mounted to a tether. The tether comprises a flexible cable. The tether is strong in tensile. The tether is soft in compression. With this embodiment the anchor wire comprises an inner lumen and said tether extends through at least a portion of said lumen. The tether limits movement of the treatment element away from the anchor. The anchor wire limits movement of the treatment element towards the anchor.

In one embodiment the anchor element is located at the distal end of the anchor wire.

In another embodiment the anchor element at least partially penetrates the heart wall in use. The device further comprises an anchor limiter. The level of penetration of the tissue wall by the anchor element is limited by the anchor limiter.

In one case the anchor limiter engages a wall of tissue at the site of anchoring. The anchor limiter may comprise a tissue engagement element. The anchor limiter may engage the surface of a tissue wall at the site of anchoring. The anchor limiter may at least partially penetrates the surface of the tissue wall at the site of anchoring. Preferably the anchor limiter is adjacent the anchor.

In one embodiment the anchor limiter is expandable. Thus, the anchor limiter has an expanded configuration and a collapsed configuration. In the expanded configuration the anchor limiter comprises at least one arm. In one case the at least one arm comprises a strut.

In one embodiment the anchor limiter is deployed from a delivery device. The material of the anchor limiter may comprise a polymer, a metal, a stainless steel, a nitinol, a shape memory material, a super elastic material, a stainless alloy.

In one embodiment the anchor limiter is a nitinol element with an expanded state and a collapsed state. The anchor limiter comprises arms extending radially outwardly in the expanded state. The anchor limiter may be manufactured from a hypotube. The anchor limiter may comprise a slotted hypotube.

In one embodiment the anchor limiter may interact with the tissue wall surface to reinforce the grip of the anchor. The anchor limiter may interact with the tissue wall and prevents the anchor from disengaging with the tissue wall. The anchor limiter may prevent the anchor from unscrewing from the tissue wall by engaging with the tissue wall. In another embodiment the anchor limiter comprises a cuff. The cuff comprises an engagement surface.

In another embodiment the treatment element comprises a hydrogel. The treatment element has a dehydrated state and a hydrated state and the dehydrated state corresponds to the collapsed delivery configuration and the hydrated state corresponds to the expanded treatment configuration. In the hydrated state the hydrogel comprises primarily of polymer and a hydrating liquid and at least some of the polymer molecules are cross-linked. In the hydrating liquid comprises saline, contrast media, a biocompatible fluid, silicone fluid, or blood plasma components. In the expanded hydrated state the hydrogel is mostly liquid with solid polymer network spanning the occupied volume.

This section 6 describes aspects of the design construction and materials of hydrogel based treatment elements. Additional features and embodiments are described in more detail in Sections 2 and 9 of this statement and are incorporated in their entirety in this section 6 by reference.

### Section 7

According to the invention there is provided a medical device suitable for use in treatment of a valve, the device comprising a treatment element, an anchor element, and a support element and an abutment stop wherein, the treatment element is configured to be located at the region of coaptation of leaflets of a valve to resist fluid flow in a retrograde direction through an opening of the valve, the anchor element configured to anchor the treatment element to a heart wall, the anchor element being located at an end of the support element, the support element extending from the anchor and supporting the treatment element at the region of coaptation, an abutment stop configured to limit movement of the treatment element relative to the anchor.

In one embodiment the abutment stop comprises an engagement surface. In another the abutment stop is connected to the anchor. In yet another, the abutment stop is integral with the support element.

The treatment element further comprises at least one engagement surface and engagement of the at least one treatment element engagement surface with the at least one abutment stop limits the axial movement of the treatment element along the support element. In one case the at least one treatment element engagement surface comprises at least one collar and said at least one collar engages with said at least one abutment stop to limits the axial movement of the treatment element along the support element.

In use the abutment stop is implanted in the patient but is preferably shielded from direct contact with flowing blood. In one case the abutment stop is shielded from direct contact with flowing blood by positioned it within the body of the treatment element. The abutment stop may be positioned between the distal end and the proximal end of the treatment element. Engagement between said abutment stop and said engagement surface occurs between the distal and proximal end of the treatment element.

The support element abutment stop may comprise a step on the outer surface of the support. The step on the outer surface of the support may comprise a collar. The collar comprises at least one abutment surface.

In another embodiment the support element abutment stop comprises a step on the inner surface of the support. The step on the inner surface of the support may comprise recess.

In another embodiment the abutment stop is mounted to a tether. The tether comprises a flexible cable that is strong in tensile. The tether may optionally be soft in compression. With this embodiment the support comprises an inner lumen and said tether extends through at least a portion of said lumen. The tether is fixed at one end to the anchor and to the stop at the other end. The tether limits movement of the treatment element away from the anchor. The support limits movement of the treatment element towards the anchor.

The anchor element may be located at the distal end of the support element. The anchor element may be located at the proximal end of the support element.

In another embodiment the device comprises an anchor wire for anchoring the anchor to the heart wall. The anchor wire is configured to transmit axial and/or torsional movements from a proximal end to the anchor at the distal end, said axial and/or torsional movements facilitating the anchoring of the anchor to the heart wall, wherein, the anchor wire comprises a coupling for transmission of said axial and torsional movements to the anchor. The anchor wire comprises a proximal end and a distal end. The proximal end of the anchor wire extends proximal of the coupling. In another embodiment the anchor wire extends distal of the coupling.

In one case the coupling comprises a pair of coupling features. The pair of coupling features comprises a proximal coupling feature and a distal coupling feature. The proximal coupling feature may be fixed to a proximal segment of the anchor wire and said distal coupling feature may be fixed to a distal segment of the anchor wire. The proximal end of the anchor wire can be separated from the distal end of the anchor wire by decoupling the proximal coupling feature from the distal coupling feature.

This section 7 describes aspects of the design, construction, materials and use of anchor wire couplings. Additional features and embodiments are described in more detail in Sections 6 and 8 of this statement and are incorporated in their entirety in this section 7 by reference.

In another embodiment the anchor element is configured to be anchored to the heart wall and said heart wall is in cyclical motion relative to said valve. The device further comprises a delivery device, the delivery device comprising a distal end, a proximal end, and configured to advance the treatment element and deploy the treatment element at the treatment location. The delivery device comprises a slack promoting region. The slack promoting region of the delivery device comprises a region wherein the delivery device has a low mechanical resistance to changes in its radius of curvature in response to compressive or tensile forces.

The slack promoting region of the delivery device in use may comprise a curved segment and the shape of said curved segment changes during the cardiac cycle. In one embodiment the delivery device comprises a delivery catheter with a reception space at the distal end to house the treatment element in a collapsed state for delivery. The slack promoting region is proximal of the reception space.

In another embodiment the delivery device comprises a support the support extending from the anchor to the treatment element. The treatment element is supported at the region of coaptation by the support. The support comprises a slack promoting region proximal of the anchor. In another case the support comprises a slack promoting region proximal of the treatment element.

This section 7 describes aspects of the design, construction, materials and methods associated with the slack promoting features. Additional features and embodiments are described in more detail in Sections 4 and 8 of this statement and are incorporated in their entirety in this section 7 by reference.

In another embodiment the device further comprises at least one mounting tube. The treatment element is mounted to at least one mounting tube. The at least one mounting tube may be rotatable relative to the support.

In one embodiment the support extends from the tissue wall and supports the treatment element at the region of coaptation. In one case the treatment element is sealingly mounted to the at least one mounting tube. In another case the at least one mounting tube is integral with the treatment element. In another embodiment the mounting tube comprises an extruded tube.

This section 7 describes aspects of mounting the treatment elements at the treatment location. Additional features and embodiments are described in more detail in Section 3 of this statement and are incorporated in their entirety in this section 7 by reference.

In another embodiment the anchor element is inserted into the wall of the heart to anchor the treatment element. The anchor at least partially penetrates the heart wall in use. The device further comprises an anchor limiter. The penetration of the tissue wall by the anchor element is limited by an anchor limiter.

In one case the anchor limiter engages a wall of tissue at the site of anchoring. The anchor limiter may comprise a tissue engagement element. The anchor limiter engages the surface of a tissue wall at the site of anchoring. In one case the anchor limiter at least partially penetrates the surface of the tissue wall at the site of anchoring.

In one embodiment the anchor limiter is adjacent the anchor. In another embodiment the anchor limiter is expandable. The expandable anchor limiter has an expanded configuration and a collapsed configuration.

In one embodiment, in the expanded configuration the anchor limiter comprises at least one arm. The at least one arm may comprise a strut. In another embodiment the anchor limiter is deployed from a delivery device. The material of the anchor limiter comprises a polymer, a metal, a stainless steel, a nitinol, a shape memory material, a super elastic material, a stainless alloy.

This section 7 describes aspects of features of anchor limiters. Additional features and embodiments are described in more detail in Section 10 of this statement and are incorporated in their entirety in this section 7 by reference.

In another embodiment the treatment element comprises a hydrogel. The treatment element has a dehydrated state and a hydrated state and the dehydrated state corresponds to the collapsed delivery configuration and the hydrated state corresponds to the expanded treatment configuration. In the hydrated state the hydrogel comprises primarily of polymer and a hydrating liquid and at least some of the polymer molecules are cross-linked. In the hydrating liquid comprises saline, contrast media, a biocompatible fluid, silicone fluid, or blood plasma components. In the expanded hydrated state the hydrogel is mostly liquid with solid polymer network spanning the occupied volume. Preferably the hydrogel comprises at least 50% liquid by volume in the hydrated state. More preferably the hydrogel comprises at least 70% liquid by volume in the expanded hydrated state. More preferably the hydrogel comprises at least 80% liquid by volume in the expanded hydrated state. In one embodiment the density of the treatment element is in the range 1200 kg/m³ to 1025 kg/m³ in the expanded state.

This section 7 describes aspects of the design construction and materials of hydrogel based treatment elements. Additional features and embodiments are described in more detail in Sections 2 and 9 of this statement and are incorporated in their entirety in this section 7 by reference.

### Section 8

The invention also provides a method of treating a valve using a treatment device, the treatment device comprising a treatment element, a support, and a delivery device, the method comprising the steps of:
advancing the treatment element, the support and the distal end of the delivery device into a heart chamber;
anchoring the support to a body wall;
advancing the delivery device so as to create slack in the delivery device in a heart chamber;
deploying the treatment element at the treatment location.

In one embodiment the method, the step of deploying the treatment element further comprises retracting at least a portion of the delivery device. The deploying step may comprise holding at least a portion of the delivery device steadfast during the deployment action. The deploying step may comprise inflating the treatment element at the treatment location.

In one variant of the method the delivery device comprises a delivery catheter with a reception space at its distal end and the step of deploying the treatment element comprises unsheathing the treatment element. The step of unsheathing the treatment element may comprise the step of retracting the delivery catheter.

In one embodiment the method includes the step of removing a portion of the delivery device from the patient. The removing step may comprise decoupling a proximal segment of the support.

In one embodiment the method includes the step of implanting a proximal segment of the support proximal of the left heart chambers. The implanting step may comprise implanting the proximal segment of the support in a septal wall, in an artery, in a vein, or in tissue external of a blood vessel. The implanting step may comprise implanting the proximal segment of the support proximal of the left ventricle. The implanting step may comprise anchoring the proximal end of the support in a tissue body. The implanting step may comprise terminating the proximal end of the support.

In one embodiment the method step of advancing the treatment element, the support and the distal end of the delivery device into a heart chamber further comprises making an intra-atrial septal puncture and advancing the treatment element, the support and the distal end of the delivery device across the atrial septum.

In one embodiment the method step of advancing the treatment element, the support and the distal end of the delivery device into a heart chamber comprises inserting the device through an atrial wall with a surgical technique, or through an atrial wall with an endovascular technique, or through the aortic valve with an endovascular approach, or through the apex of the ventricle with a surgical technique.

In one embodiment the method includes the step of advancing at least a portion of the support across the valve leaflets. In one case the method includes the step of advancing at least a portion of the treatment element across the valve. This step may further comprise the step of steering the delivery device as the treatment element is advanced.

The method may comprise the step of advancing at least a portion of the support across the valve. This step may further comprise the step of steering the delivery device as the support is advanced.

In one embodiment the step of anchoring the support to a body wall comprises advancing the support relative to at least a portion of the delivery device. In one case the method the step anchoring the support to a body wall comprises advancing the support relative to the treatment element. In another case the anchoring step involves providing a support, the support comprising an inner shaft and an outer shaft and the anchoring step comprises moving the inner shaft relative to the outer shaft to anchor the support to the tissue wall. This step may further comprise providing an expandable anchor element and the step of anchoring the anchor in the wall of body tissue comprises advancing the anchor into a tissue wall and expanding the anchor within the tissue wall. The method may comprise the step of imaging the delivery device. The method may also comprise the step of sizing the treatment element to the regurgitant orifice. The step of sizing the treatment element to the regurgitant orifice may comprise controllably injecting a volume of fluid into a fluid sac of the treatment element.

In one embodiment the method comprises the step of decoupling at least a portion of the delivery device and removing the decoupled portion from the patient. The decoupling step may comprise decoupling at least a portion of the delivery device from the support and removing the decoupled portion from the patient. The decoupling step may comprise decoupling at least a portion of the delivery device from the treatment element and removing the decoupled portion from the patient. The decoupling step may comprise decoupling at least a proximal portion of the support from the distal portion of the support and removing the decoupled portion from the patient.

### Section 9

According to the invention there is further provided a medical device suitable for use in treatment of a valve, the device comprising a treatment element, the treatment element configured to be located at the region of coaptation of leaflets of a valve to resist fluid flow in a retrograde direction through an opening of the valve, the treatment element comprising a collapsed delivery configuration and an expanded treatment configuration, wherein the treatment element comprises a hydrogel.

In one embodiment the treatment element further comprises a support for supporting the treatment element at the region of coaptation, the support being connectable to a wall of body tissue. The wall of body tissue comprises a ventricle wall, an atrial wall, a septal wall, or a vessel wall.

The support comprises an anchor for fixing the support to the wall of body tissue. The treatment element has a dehydrated state and a hydrated state and the dehydrated state corresponds to the collapsed delivery configuration and the hydrated state corresponds to the expanded treatment configuration. In the hydrated state the hydrogel comprises primarily of polymer and a hydrating liquid and at least some of the polymer molecules are cross-linked. In the hydrating liquid comprises saline, contrast media, a biocompatible fluid, silicone fluid, or blood plasma components. In the expanded hydrated state the hydrogel is mostly liquid with solid polymer network spanning the occupied volume. Preferably the hydrogel comprises at least 50% liquid by volume in the hydrated state. More preferably the hydrogel comprises at least 70% liquid by volume in the expanded hydrated state. More preferably the hydrogel comprises at least 80% liquid by volume in the expanded hydrated state. More preferably the hydrogel comprises at least 90% liquid by volume in the expanded hydrated state. More preferably the hydrogel comprises at least 95% liquid by volume in the expanded hydrated state. Most preferably the hydrogel comprises from 95% to 99% liquid by volume in the expanded hydrated state.

In another embodiment the density of the treatment element is in the range 1200 kg/m³ to 1025 kg/m³ in the expanded state.

In another case the polymer network of the hydrogel is at least partially composed of a synthetic polymer, a protein or a natural polymer.

In another variant the compliance of the treatment element in the expanded hydrated state is greater than the compliance of the treatment element in the collapsed state.

In one embodiment the polymer network of the hydrogel is loaded with an active compound which is eluted from the hydrogel over time. The eluted compound is selected from one or more of an anticoagulant, an anti-thrombin, an anti-platelet, an agent to prevent thrombosis, an anti-proliferative, an anti-fibrotic, an agent to promote endothelialisation, and a drug. The compound comprises heparin or a factor Xa inhibitor.

In another embodiment the hydrogel is porous. In another embodiment the hydrogel is at least partially solid.

In a further embodiment the hydrogel comprises hydrophilic chain segments. The hydrophilic chain segments comprise high electronegativity atoms and said high electronegativity atoms comprise at least 10% of the atomic mass of the chain segment. The hydrophilic chain segments comprise high electronegativity atoms and said high electronegativity atoms comprise at least 20% of the atomic mass of the chain segment. The hydrophilic chain segments comprise high electronegativity atoms and said high electronegativity atoms comprise at least 25% of the atomic mass of the chain segment. The hydrophilic chain segments comprise high electronegativity atoms and said high electronegativity atoms comprise at least 30% of the atomic mass of the chain segment.

In another embodiment the polymer of the hydrogel is based on one or more of polyvinyl alcohol (PVA), sodium polyacrylate, hydrophilic acrylate polymers, hydrophilic polymethacrylates, 2-hydroxyethyl-methacrylate (HEMA), ethylene glycol bismethacrylate, hyluronan polymers, poly(anhydride esters), poly(vinylpyrroldine), poly(ethyloxazoline), poly(ethylene glycol)-co-poly(propylene glycol) block copolymers, hydrophilic methacrylamides, and a polyethylene glycol based polyurethane.

In another embodiment the treatment element is sonolucent. It will be appreciated that the sonolucent properties of the treatment element allow treatment element to be visualised on echocardiography.

In another case the hydrating liquid at least comprises a contrast medium and this allows the device to be visualised using X-ray systems including fluoroscopy.

In another embodiment the device further comprises an anchor element, a support and an abutment stop wherein, the anchor element is configured to anchor the treatment element to a heart wall, the support extending from the anchor and supporting the treatment element at the region of coaptation, and the abutment stop configured to limit movement of the treatment element relative to the anchor.

In one embodiment the abutment stop comprises an engagement surface. The abutment stop is preferably connected to the anchor. The abutment stop may be integral with the support. The treatment element further comprises at least one engagement surface and engagement of the at least one treatment element engagement surface with the at least one abutment stop limits the axial movement of the treatment element along the support element.

In one case the at least one treatment element engagement surface comprises at least one collar and said at least one collar engages with said at least one abutment stop to limits the axial movement of the treatment element along the support element. In use the abutment stop is implanted in the patient but is preferably shielded from direct contact with flowing blood. The abutment stop may be shielded from direct contact with flowing blood by positioned it within the body of the treatment element. The abutment stop may be positioned between the distal end and the proximal end of the treatment element.

This section 9 describes features of mounting and controlling the movement of the treatment elements at the treatment location. Additional features and embodiments are described in more detail in Section 7 of this statement and are incorporated in their entirety in this section 9 by reference.

In another embodiment the device further comprises an anchor element and an anchor wire wherein, the anchor element is configured to anchor the treatment element to a heart wall, the anchor wire configured for anchoring the anchor to the heart wall.

The anchor wire is configured to transmit axial and/or torsional movements from a proximal end to the anchor at the distal end, said axial and/or torsional movements facilitating the anchoring of the anchor to the heart wall, wherein, the anchor wire comprises a coupling for transmission of said axial and torsional movements to the anchor. The anchor wire comprises a proximal end and a distal end.

The proximal end of the anchor wire extends proximal of the coupling. The distal end of the anchor wire extends distal of the coupling. The coupling may comprise a pair of coupling features. The pair of coupling features comprises a proximal coupling feature and a distal coupling feature.

In one embodiment the proximal coupling feature is fixed to a proximal segment of the anchor wire and said distal coupling feature is fixed to a distal segment of the anchor wire. The proximal end of the anchor wire can be separated from the distal end of the anchor wire by decoupling the proximal coupling feature from the distal coupling feature.

This section 9 describes features associated with the anchor wire and anchor wire coupling. Additional features and embodiments are described in more detail in Section 6 of this statement and are incorporated in their entirety in this section 9 by reference.

In another embodiment the device further comprises an anchor element wherein the anchor element is configured to be anchored to the heart wall and said heart wall is in cyclical motion relative to said valve. The device further comprises a delivery device, the delivery device comprising a distal end, a proximal end, and configured to advance the treatment element and deploy the treatment element at the treatment location. The delivery device comprises a slack promoting region.

The slack promoting region of the delivery device comprises a region wherein the delivery device has a low mechanical resistance to changes in its radius of curvature in response to compressive or tensile forces.

In one embodiment the slack promoting region of the delivery device in use comprises a curved segment and the shape of said curved segment changes during the cardiac cycle.

In another embodiment the delivery device comprises a delivery catheter with a reception space at the distal end to house the treatment element in a collapsed state for delivery. The slack promoting region is proximal of the reception space.

In another embodiment the delivery device comprises a support the support extending from the anchor to the treatment element. The treatment element is supported at the region of coaptation by the support. The support comprises a slack promoting region proximal of the anchor.

This section 9 describes aspects of delivering the treatment elements to the treatment location. Additional features and embodiments are described in more detail in Section 4 of this statement and are incorporated in their entirety in this section 9 by reference.

In another embodiment the device further comprises a support and at least one mounting tube. The treatment element is mounted to at least one mounting tube. The at least one mounting tube may be rotatable relative to the support.

In one embodiment the support extends from the tissue wall and supports the treatment element at the region of coaptation. In another embodiment the treatment element is sealingly mounted to the at least one mounting tube. In yet another embodiment the at least one mounting tube may be integral with the treatment element.

In one case the mounting tube comprises an extruded tube. The material of the mounting may be a polymer, or a metal. The material of the mounting may be a biocompatible polymer or metal.

In another embodiment the mounting of the treatment element on the mounting tube comprises an interface layer. The interface layer comprises a mixture of mounting tube material and treatment element material. The interface layer is at least partially resistant to fluid flow.

This section 9 describes aspects of mounting the treatment elements at the treatment location. Additional features and embodiments are described in more detail in Section 3 of this statement and are incorporated in their entirety in this section 9 by reference.

In another embodiment the device further comprises an anchor wire and an anchor element wherein the anchor element anchors the treatment element to a wall of tissue and in use the anchor element at least partially penetrates the tissue wall.

The device further comprises an anchor limiter. The penetration of the tissue wall by the anchor element is limited by an anchor limiter. The anchor limiter engages a wall of tissue at the site of anchoring. The anchor limiter comprises a tissue engagement element.

In one embodiment the anchor limiter at least partially penetrates the surface of the tissue wall at the site of anchoring. Preferably the anchor limiter is adjacent the anchor.

In another embodiment the anchor limiter is expandable. In this case the anchor limiter has an expanded configuration and a collapsed configuration. In the expanded configuration the anchor limiter comprises at least one arm. The at least one arm may comprise a strut.

In one embodiment the anchor limiter is deployed from a delivery device. In another embodiment the anchor limiter is a nitinol element with an expanded state and a collapsed state. The anchor limiter comprises arms extending radially outwardly in the expanded state. The struts of the anchor limiter lie along the anchor wire in the collapsed state. The anchor limiter may be manufactured from a hypotube. The anchor limiter comprises a slotted hypotube. The anchor limiter may be made from wire.

In another embodiment the anchor limiter interacts with the tissue wall surface to reinforce the grip of the anchor. The anchor limiter may interact with the tissue wall and prevents the anchor from disengaging from the tissue wall. In another embodiment the anchor limiter prevents the anchor from unscrewing from the tissue wall.

In another embodiment the anchor limiter comprises a cuff. The cuff comprises an engagement surface.

### Section 10

According to the invention there is further provided a medical device suitable for use in treatment of a valve, the device comprising a treatment element, an anchor element, and an anchor limiter wherein, the treatment element is configured to be located at the region of coaptation of leaflets of a valve to resist fluid flow in a retrograde direction through an opening of the valve, the anchor element configured to anchor the treatment element to a tissue wall, the anchor element comprising an anchor limiter.

In use the anchor penetrates the tissue wall to anchor the treatment element at the treatment location. The penetration of the tissue wall by the anchor element is limited by an anchor limiter. The anchor limiter engages a wall of tissue at the site of anchoring. The anchor limiter comprises a tissue engagement element.

In one embodiment the anchor limiter at least partially penetrates the surface of the tissue wall at the site of anchoring. In another embodiment the anchor limiter is adjacent the anchor.

In yet another embodiment the anchor limiter is expandable. The anchor limiter has an expanded configuration and a collapsed configuration. In the expanded configuration the anchor limiter engages body tissue. The anchor limiter is delivered in the collapsed state.

In one embodiment, in the expanded configuration the anchor limiter comprises at least one arm. Preferably the at least one arm comprises a strut. In another embodiment the anchor limiter is deployed from a delivery device. The material of the anchor limiter comprises a polymer, a metal, a stainless steel, a nitinol, a shape memory material, a super elastic material, a stainless alloy.

In one embodiment the anchor limiter is a nitinol element with an expanded state and a collapsed state. The anchor limiter comprises arms extending radially outwardly in the expanded state.

In one embodiment the anchor limiter is manufactured from a hypotube. In one case the anchor limiter comprises a slotted hypotube. In another case the anchor limiter comprises a wire element.

In another embodiment the anchor limiter interacts with the tissue wall surface to reinforce the grip of the anchor. The anchor limiter interacts with the tissue wall and prevents the anchor from disengaging with the tissue wall. In one case the anchor limiter prevents the anchor from unscrewing from the tissue wall.

In another embodiment the anchor limiter comprises a cuff. The cuff comprises an engagement surface.

In another embodiment the treatment element comprises a hydrogel. The treatment element further comprises a support for supporting the treatment element at the region of coaptation, the support being connected to the anchor element. The wall of tissue wall comprises a ventricle wall, an atrial wall, a septal wall, or a vessel wall.

In one embodiment the support comprises an anchor for fixing the support to the wall of body tissue.

The treatment element has a dehydrated state and a hydrated state and the dehydrated state corresponds to the collapsed delivery configuration and the hydrated state corresponds to the expanded treatment configuration. In the hydrated state the hydrogel comprises primarily of polymer and a hydrating liquid and at least some of the polymer molecules are cross-linked. The hydrating liquid may comprise saline, contrast media, a biocompatible fluid, silicone fluid, or blood plasma components. In the expanded hydrated state the hydrogel is mostly liquid with solid polymer network spanning the occupied volume.

This section 10 describes aspects of the design construction and materials of hydrogel based treatment elements. Additional features and embodiments are described in more detail in Sections 2 and 9 of this statement and are incorporated in their entirety in this section 10 by reference.

In another embodiment the device further comprises a support and an abutment stop wherein, the support extends from the anchor and supports the treatment element at the region of coaptation, and the abutment stop is configured to limit movement of the treatment element relative to the anchor.

In one embodiment the abutment stop comprises an engagement surface. The abutment stop is connected to the anchor. The abutment stop is integral with the support.

In another embodiment the treatment element further comprises at least one engagement surface and engagement of the at least one treatment element engagement surface with the at least one abutment stop limits the axial movement of the treatment element along the support element. The at least one treatment element engagement surface comprises at least one collar and said at least one collar engages with said at least one abutment stop to limits the axial movement of the treatment element along the support element.

In use the abutment stop is implanted in the patient but is preferably shielded from direct contact with flowing blood. The abutment stop may be shielded from direct contact with flowing blood by positioned it within the body of the treatment element. The abutment stop may be positioned between the distal end and the proximal end of the treatment element.

This section 10 describes features of mounting and controlling the movement of the treatment elements at the treatment location. Additional features and embodiments are described in more detail in Section 7 of this statement and are incorporated in their entirety in this section 10 by reference.

In another embodiment the device further comprises an anchor wire wherein, the anchor wire is configured for anchoring the anchor to the heart wall. The anchor wire is configured to transmit axial and/or torsional movements from a proximal end to the anchor at the distal end, said axial and/or torsional movements facilitating the anchoring of the anchor to the heart wall, wherein, the anchor wire comprises a coupling for transmission of said axial and torsional movements to the anchor. The anchor wire comprises a proximal end and a distal end. The proximal end of the anchor wire extends proximal of the coupling. The distal end of the anchor wire extends distal of the coupling.

In one embodiment the coupling comprises a pair of coupling features.

This section 10 describes features associated with the anchor wire and anchor wire coupling are partially described in this section. Additional features and embodiments are described in more detail in Section 6 of this statement and are incorporated in their entirety in this section 10 by reference.

In another embodiment the anchor element is configured to be anchored to the heart wall while said heart wall is in cyclical motion relative to said valve. The device further comprises a delivery device, the delivery device comprising a distal end, a proximal end, and configured to advance the treatment element and deploy the treatment element at the treatment location. The delivery device comprises a slack promoting region. The slack promoting region of the delivery device comprises a region wherein the delivery device has a low mechanical resistance to changes in its radius of curvature in response to compressive or tensile forces.

In one embodiment the slack promoting region of the delivery device in use comprises a curved segment and the shape of said curved segment changes during the cardiac cycle.

In another embodiment the delivery device comprises a delivery catheter with a reception space at the distal end to house the treatment element in a collapsed state for delivery. The slack promoting region is proximal of the reception space.

In yet another embodiment the delivery device comprises a support the support extending from the anchor to the treatment element. The treatment element is supported at the region of coaptation by the support. In one case the support comprises a slack promoting region proximal of the anchor. In another case the support comprises a slack promoting region proximal of the treatment element.

This section 10 describes aspects of delivering the treatment elements to the treatment location. Additional features and embodiments are described in more detail in Section 4 of this statement and are incorporated in their entirety in this section 10 by reference.

In another embodiment the device further comprises a support and at least one mounting tube. The treatment element is mounted to at least one mounting tube. In one embodiment the at least one mounting tube may be rotated relative to the support. The support extends from the anchor element and supports the treatment element at the region of coaptation.

In another embodiment the treatment element is sealingly mounted to the at least one mounting tube. The at least one mounting tube may be integral with the treatment element. The mounting tube comprises an extruded tube. The material of the mounting tube is preferably a polymer, or a metal.

This section 10 describes aspects of mounting the treatment elements at the treatment location. Additional features and embodiments are described in more detail in Section 3 of this statement and are incorporated in their entirety in this section 10 by reference.

### BRIEF DESCRIPTION OF THE DRAWINGS

The invention will be more clearly understood from the following description of some embodiments thereof, given by way of example only, with reference to the accompanying drawings, in which

FIG. 1 is a side view of a medical device according to the invention, in use;

FIG. 2 is a cross-sectional, side view of the device of FIG. 1;

FIG. 3 is a cross-sectional, end view of the device of FIG. 1 in a second treatment configuration;

FIG. 4 is a cross-sectional, side view of another medical device according to the invention;

FIG. 5 is a cross-sectional, end view of the device of FIG. 4 in a first treatment configuration;

FIG. 6 is a cross-sectional, end view of the device of FIG. 4 in a second treatment configuration;

FIG. 7 is a cross-sectional, end view of the device of FIG. 4 in an alternative second treatment configuration;

FIGS. 8 to 10 are views similar to FIGS. 4 to 6 of another medical device according to the invention;

FIGS. 11 to 14 are views similar to FIGS. 4 to 7 of another medical device according to the invention;

FIGS. 15 to 18 are views similar to FIGS. 4 to 7 of a further medical device according to the invention;

FIG. 19 is a cross-sectional view of another medical device according to the invention;

FIG. 20 is a cross-sectional view of another medical device according to the invention;

FIG. 21 is a cross-sectional view of another medical device according to the invention;

FIG. 22 is a cross-sectional view of another medical device according to the invention;

FIG. 23 is a cross-sectional view of another medical device according to the invention;

FIG. 24 is a cross-sectional view of another medical device according to the invention;

FIG. 25 is a cross-sectional view of another medical device according to the invention;

FIG. 26 is a cross-sectional view of another medical device according to the invention;

FIG. 27 is a cross-sectional view of another medical device according to the invention;

FIG. 28 is a cross-sectional view of another medical device according to the invention;

FIG. 29 is a cross-sectional view of another medical device according to the invention with the treatment element in an expanded configuration;

FIG. 30 is a cross-sectional view of the medical device of FIG. 29 according to the invention. The treatment element is in a collapsed configuration;

FIG. 31 is a side view of a delivery catheter according to the invention;

FIG. 32 is a side view of another delivery catheter according to the invention;

FIG. 33 is a side view of another delivery catheter according to the invention;

FIG. 34 is a cross sectional side view of a device of the invention in a delivery configuration;

FIG. 35 is a cross sectional side view of another device of the invention in a delivery configuration;

FIG. 36 is a cross sectional view of the left heart chambers with a device of the invention implanted;

FIG. 37 is a side cross-sectional view of another medical device according to the invention;

FIG. 38 is a side cross-sectional view of another medical device according to the invention;

FIG. 39 is another cross-sectional view of the medical device of FIG. 38 with the hinged anchor offset relative to the support;

FIG. 40 is a side cross-sectional view of another medical device according to the invention;

FIG. 41 is a side cross-sectional view of another medical device according to the invention;

FIG. 42 is a side cross-sectional view of another medical device according to the invention;

FIG. 43a is a cross-sectional view showing a device of the invention being advanced into a heart chamber across a valve;

FIG. 43b is a cross-sectional view showing a device of the invention being anchored to a tissue wall;

FIG. 43c is a cross-sectional view of a device of the invention in a heart chamber with a slack promoting feature;

FIG. 43d is a cross-sectional view of a device of the invention in a heart chamber with the treatment element being deployed;

FIG. 43e is a cross-sectional view of a device of the invention in a heart chamber with at least a portion of a delivery device removed;

FIG. 43f is a cross-sectional view of a device of the invention in a heart chamber with the treatment element expanded between valve leaflets; and

FIG. 43g is a cross-sectional view of a device of the invention implanted in a heart chamber.

### DETAILED DESCRIPTION

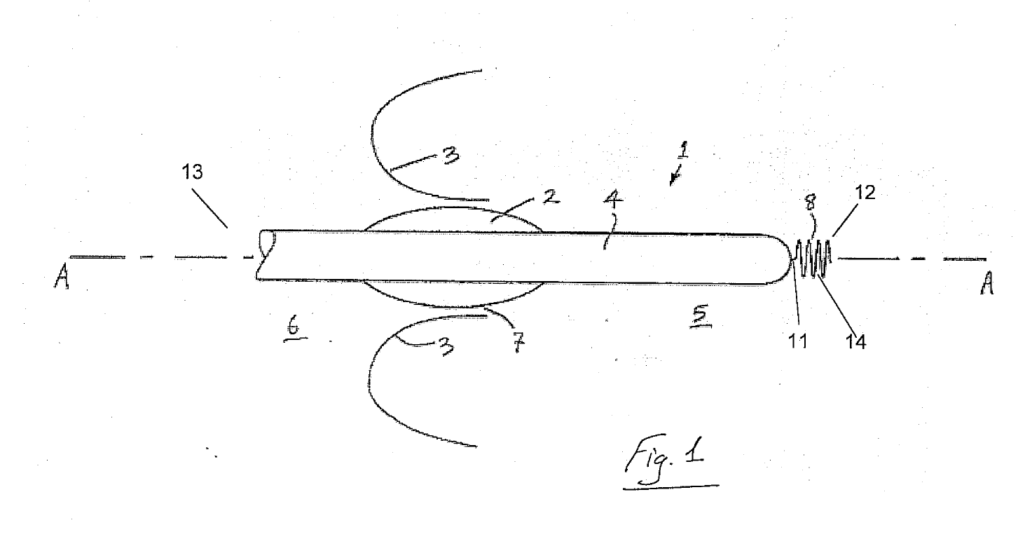

Referring to the drawings, and initially to FIGS. 1 to 3 thereof, there is illustrated a medical device 1 according to the invention. The device 1 is suitable for use in treatment of a valve, for example for treatment of one of the atrioventricular heart valves, such as the mitral valve.

The device 1 comprises a treatment element 2 which is configured to be located at the region of co-aptation of the leaflets 3 of the atrioventricular heart valve, a support element 4 which supports the treatment element 2 at the region of co-aptation of the valve leaflets 3 (FIG. 1), and an anchor element 8 to anchor the support element 4 to the wall of the ventricle 5.

The treatment element 2 acts to resist blood flow in the retrograde direction from the ventricle 5 into the atrium 6 through the valve opening 7.

The treatment element 2 comprises a porous hydrogel material, in this case (FIG. 2). Upon contact of blood with the treatment element 2, the treatment element 2 expands from a low-profile storage configuration to a first treatment configuration.

For the purposes of this invention a hydrogel shall mean an apparently solid, jelly-like material which is at least partially composed of a polymer molecule. The hydrogel has a hydrated state and a de-hydrated state and a partially hydrated state. In the hydrated state the hydrogel comprises primarily of polymer and a hydrating liquid. The polymer consists of long chain molecules. Preferably at least some of the polymer molecules are cross-linked. More preferably the polymer molecules are cross-linked to form a three dimensional molecular network. The hydrogel assumes a lesser volume in the de-hydrated state and occupies more volume in the expanded state. The hydrogel is hydrated with a liquid. A variety of hydrating liquids are possible. Saline, contrast media and blood plasma components include some potential hydrating liquid media. In the expanded hydrated state the hydrogel is mostly liquid with solid polymer network spanning the occupied volume. In the hydrated state the hydrogel behaves like a solid due to the polymer network. Preferably the hydrogel is composed of over 50% liquid in the hydrated state. More preferably the hydrogel is composed of over 70% liquid in the expanded hydrated state. More preferably the hydrogel is composed of over 80% liquid in the expanded hydrated state. More preferably the hydrogel is composed of over 90% liquid in the expanded hydrated state.

More preferably the hydrogel is composed of over 95% liquid in the expanded hydrated state. More preferably the hydrogel is composed of between 95% and 99% liquid in the expanded hydrated state.

Preferably the density of the treatment element is close to the density of blood in the expanded state. This is a particular advantage of this design as the majority of the mass of the treatment device is absorbed from blood surrounding the device. Preferably the density of the treatment element is closer to the density of blood in the expanded state than to the density of the de-hydrated hydrogel.

In one embodiment the polymer network of the hydrogel is at least partially composed of a synthetic polymer. In another embodiment the polymer network of the hydrogel is at least partially composed of a protein. In another embodiment the polymer network of the hydrogel is at least partially composed of natural polymer.

Preferably the compliance of the treatment element in the expanded hydrated state is close to the compliance of soft body tissue.

In another embodiment the hydrogel is loaded with an active molecule which is eluted over time. In one embodiment the active molecule is a chemoattractant. In another the active molecule is a drug.

In one embodiment the hydrogel is porous. In another embodiment the hydrogel is at least partially solid.

The polymer network of the hydrogel is at least partially composed of a hydrophilic polymer. A hydrophilic molecule or portion of a molecule is one that is typically charge-polarized and capable of hydrogen bonding, enabling it to dissolve more readily in water than in oil or other hydrophobic solvents.

Hydrophilic and hydrophobic molecules are also known as polar molecules and nonpolar molecules, respectively. Polymer molecules become polarized due to differences in the electronegativity of the atoms that make up the molecule. High electronegativity elements such as Oxygen, Nitrogen, and Chlorine exert a strong pull on shared electrons in the covalent bonds of the polymer and thus create a polar charge in their immediate vicinity. If the polymer has a high concentration of this type of bond then it will display hydrophilic properties.

In general hydrogel properties are displayed where the polymer network has concentrations of elements with high electronegativity. For the purpose of this invention high electronegativity atoms shall mean atoms with an electronegativity value equal to or greater than 3.0 on the Pauling Electronegativity Scale. Electronegativity tables are readily available in the literature. A number of electronegativity scales are available. For the purposes of this invention the Pauling electronegativity scale will be used. Below is a list of important elements in the construction of hydrogel polymers. The electronegativity value of each element is in brackets.

Hydrogen (2.1); Oxygen (3.44); Nitrogen (3.04); Chlorine (3.16); Carbon (2.55);

It will be noted that Oxygen has a very high electronegativity number and is a very important element in building hydrogel networks. On the other hand the polymer network needs to have sufficient hydrocarbon elements to retain structure.

In a preferred embodiment of this invention the hydrogel polymer of the treatment element comprises a network comprising a hydrocarbon component and a concentration of high electronegativity elements. Preferably the high electronegativity elements have electronegativity values of 3.0 or greater. In one embodiment the high electronegativity elements comprise at least 10% of the molecular mass of the polymer network. In another embodiment the high electronegativity elements comprise at least 20% of the molecular mass of the polymer network. In another embodiment the high electronegativity elements comprise at least 25% of the molecular mass of the polymer network. In another embodiment the high electronegativity elements comprise at least 30% of the molecular mass of the polymer network.

In one embodiment the hydrogel material comprises hydrophilic chain segments and hydrophobic chain segments. Preferably the hydrophilic chain segments comprise a concentration of high electronegativity elements. Preferably the high electronegativity elements of the hydrophilic chain segment have electronegativity values of 3.0 or greater. In one embodiment the high electronegativity elements of the hydrophilic chain segment comprise at least 10% of the molecular mass of the hydrophilic chain segment. In another embodiment the high electronegativity elements of the hydrophilic chain segment comprise at least 20% of the molecular mass of the hydrophilic chain segment. In another embodiment the high electronegativity elements of the hydrophilic chain segment comprise at least 25% of the molecular mass of the hydrophilic chain segment. In another embodiment the high electronegativity elements of the hydrophilic chain segment comprise at least 30% of the molecular mass of the hydrophilic chain segment.

Suitable hydrogels include hydrogels based on: polyvinyl alcohol (PVA), sodium polyacrylate, hydrophilic acrylate polymers, hydrophilic poly methacrylates, 2-hydroxyethyl-methacrylate (HEMA), ethylene glycol bismethacrylate, hyluronan polymers, poly(anhydride esters), poly(vinylpyrroldine), poly(ethyloxazoline), poly(ethylene glycol)-co-poly(propylene glycol) block copolymers, hydrophilic meth acrylamides, polyethylene glycol based polyurethanes, and other polymers and copolymers with an abundance of hydrophilic groups. It will be appreciated that the above list of hydrogel polymers is by no means exhaustive and many other potential materials could be applied to this invention.

In one embodiment the treatment element is visible under multiple imaging modalities. The gel like nature of the hydrogel makes it sonolucent and this allows the shape of the treatment element to be imaged using ultrasound imaging techniques such as trans-esophageal echocardiogram (TEE).

The treatment element of the device can also be imaged by the following method. The hydrogel component of the treatment element is partially hydrated with a contrast media. The treatment device is delivered in the collapsed state. The device is anchored to the wall of the ventricle using an anchor at the distal end of the shaft. The treatment element is expanded. Expansion of the treatment element is achieved by hydration of the hydrogel with blood fluids. At least a portion of the contrast media is retained in the structure of the hydrogel and the retained contrast media allows at least one image of the device to be captured with a fluoroscope.

In another embodiment the treatment element comprises a first hydrogel and a second hydrogel. The first hydrogel has a higher swelling ratio than the second hydrogel. With this embodiment the second hydrogel may be employed to fix the treatment device to the support element. Because the second hydrogel swells less than the first hydrogel it has greater mechanical integrity and can more strongly fix the treatment device to the support element 4. The first hydrogel on the other hand absorbs a higher ratio of fluid and is therefore important for the expansion of the treatment element. In one embodiment the first hydrogel comprises an inner layer and the second hydrogel an outer layer. In another embodiment the first hydrogel comprises the outer layer and the second hydrogel comprises the inner layer.

In yet another embodiment all of the blood contacting surfaces of the treatment device 1 comprise a hydrogel. Typically the blood contacting surfaces include at least the treatment element 2, and the support element 4. In some embodiments the anchor wire 11, the mounting tube 19, the coupling elements 49,50 and the mounting stops 24,25 may also be blood contacting and at least partially comprise a hydrogel. Since hydrogel materials absorb high percentages of water they present very biocompatible surfaces. Preferably the support comprises an inner member and an outer covering and the outer covering is at least partially a hydrogel. In one embodiment the outer covering comprises two layers, an inner hydrophobic polymer layer and an outer hydrogel layer. In one embodiment the treatment element comprises a two layer system. In this case the reinforcement layer comprises a hydrophobic polymer layer and the outer layer comprises a hydrogel.

In another embodiment the treatment element is loaded with functional molecules. In one embodiment the treatment element is loaded with an anti-infective element. Silver particles are one anti-infective agent and particles may be loaded into the body of the treatment element. In another embodiment the treatment element is loaded with a radiopaque agent. The radiopaque agent may comprise a contrast media dissolved in the matrix of the hydrogel or it may be metal particles loaded into the bulk of the polymer.

In another embodiment the treatment element is configured to elute an active molecule. The hydrogel may be loaded with the active molecules or they may be contained in a reservoir within the body of the hydrogel. Where a lumen extends from the reservoir back to the user the reservoir may be replenished by the user. This can be filled by injection of the drug into the well. The drugs that can be eluted from the treatment element include anticoagulants such as heparin, factor Xa inhibitors, direct antithrombins or antiplatelet agents to prevent device thrombosis. Other drugs that may be used include anti-proliferative and anti-fibrotic agents to prevent proliferation of endothelial or smooth muscle cells along the interface between the device and the valve endothelium.

Alternatively the device will be coated with an antibody to encourage endothelialization of the device to prevent thrombosis.

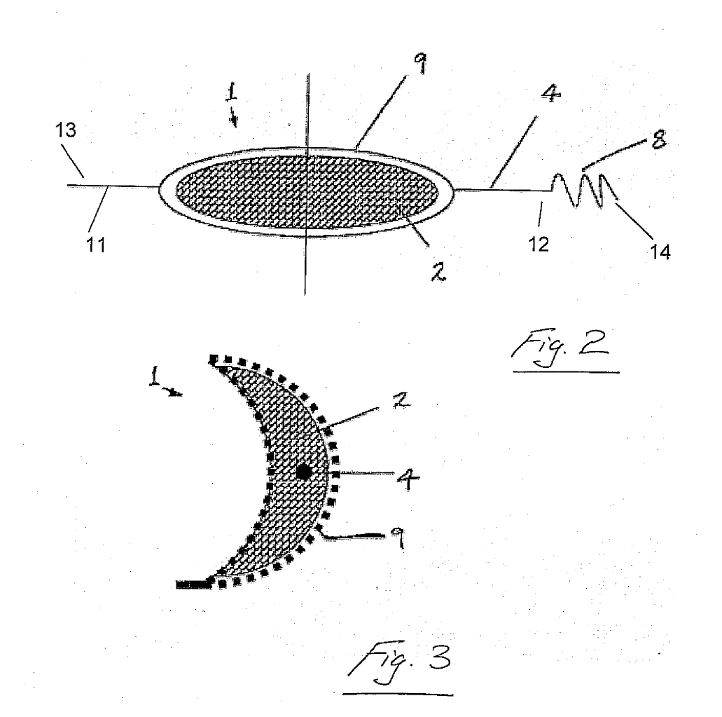

The treatment element 2 is movable in a plane perpendicular to the longitudinal axis A-A extending through the valve opening 7 between the first treatment configuration and a second treatment configuration. In the first treatment configuration, the treatment element 2 has a circular-shape in lateral cross-section. The treatment element 2 may have a diameter in the range of from 7 mm to 11 mm in the first treatment configuration. In the second treatment configuration, the treatment element 2 has a crescent-shape in lateral cross-section (FIG. 3), which approximates the shape of the mitral valve opening 7 such that the treatment element 2 substantially fills the mitral valve opening 7. The leaflets 3 of the valve engage with the treatment element 2 to move the treatment element 2 from the first treatment configuration to the second treatment configuration.

In the second treatment configuration, the treatment element 2 has the crescent-shape in lateral cross-section. The shape of the treatment element 2 is thus particularly suitable for treating the mitral valve which has the crescent-shaped opening 7.

The device 1 further comprises a reinforcement element 9 to reinforce the treatment element 2 (FIG. 2). The reinforcement element 9 comprises a braided fibre material, in this case. The reinforcement element 9 extends around the full circumference of the treatment element 2.

In this case the support element 4 is provided in the form of a flexible wire, for example a pacing lead. The support element 4 extends through the valve opening 7, in use (FIG. 1).

The treatment element 2 is fixedly attached to the support element 4. The support element 4 is advanced, in use, to deliver the treatment element 2 to the region of co-aptation of the valve leaflets 3.

The anchor element 8 is located at the distal end of the support element 4. The anchor element 8 comprises a threaded screw. The anchor element 8 may be releasably attached to the ventricle septal wall at the apex of the ventricle 5, for example by screwing the anchor element 8 into the ventricle wall. In this manner the support element 4 will be anchored to the ventricle wall and the treatment element 2 will be maintained in the desired position relative to the valve leaflets 3. The anchor element 8 extends only partially through the ventricle wall from the interior side of the ventricle wall.

The anchor element 8 further comprises an anchor wire 11. The anchor wire 11 comprises a distal end 12 and a proximal end 13. The anchor wire 11 is preferably at least partially made from a metallic material. Suitable metals for the anchor wire include stainless steel, nitinol, MP35N, and L605. The distal end 12 of anchor wire 11 comprises an anchor feature 14. In the embodiment shown the anchor feature 14 is a screw in coil. The anchor feature 14 is locked to the wall using a cork screw action. The step of anchoring the anchor element 8 to the wall of the heart involves moving the anchor relative to the heart wall. In one embodiment the shaft 4 is moved together with the anchor element 8 so as to anchor the treatment element to the heart wall. In another embodiment the anchor element is moved relative to the shaft 4 so as to anchor the anchor element 8 to the ventricle wall. In one embodiment the relative movement associated with anchoring involves a rotational movement. In another embodiment the relative movement associated with anchoring involves an axial relative movement. In another embodiment the relative movement associated with anchoring involves a combination of axial and rotational movements.

Where the fiber is a nitinol fiber the shape of the outer braid 9 in the expanded configuration can be programmed into the outer braid 9. This shape will be remembered by the outer braid 9 and this will create a bias in the device towards a particular shape. Even thought the hydrogel 2 is deformable it also has a preferred shape when hydrated in the absence of other forces. Preferably the remembered shape of the hydrogel 2 and the outer braid 9 correspond to one another.

In one embodiment the anchor element 8 is integral with the shaft 4. In another embodiment the anchor wire 11 of the anchor element 8 extends through the shaft 4 and extends back to the user and can be moved by the user so as to anchor the anchor element to a body wall.

The proximal end of the support element 4 is unconstrained relative to the wall of the ventricle 5 or the wall of the atrium 6. The proximal end of the support element 4 is located externally of the heart, in use.

A steerable delivery catheter 15 may be employed to house the treatment element 2 in the storage configuration, the support element 4 and the anchor element 8 to facilitate delivery of the treatment element 2 to the region of co-aptation of the valve leaflets 3. The treatment element 2 may be wrapped down to a low-profile while housed in the delivery catheter 15. The steerable delivery catheter may be steered in between 1 and 4 planes. The steering of the delivery catheter may be achieved by tensioning a steering cable 16. The steering cable 16 is fixed to a wall of the delivery catheter 15 near its distal end. The distal end of the catheter is configured to be more compressible than the proximal section. Applying a tension force to the proximal end of the steering cable 16 causes a compression force to act on one side of the catheter 15. This offset compression force causes the distal end of the delivery catheter tip to adopt a curved configuration and this curved configuration is used to steer the delivery catheter 15.

FIG. 2 illustrates the outer braid 9, the wire 4, the anchor 8, and the soft conformable filler 2, for example a gel or a hydrogel. In FIG. 2 the shaft 4 comprises an anchor wire 11 and the treatment element 2 is directly mounted on the wire 4.

In another embodiment device 1 comprises an expandable body 2 and a reinforcement layer 9. With this embodiment the reinforcement layer 9 at least partially restrains the expandable body at the region of coaptation. The reinforcement layer 9 is connected to the anchor 8 by the support element 4. While the reinforcement layer 9 is a flexible layer it is rigidly connected to the support element at least one point. In one embodiment the reinforcement layer 9 is rigidly fixed to the support element 4 at least one point. Preferably the reinforcement layer 9 is connected to the support element 4 at more than one point. Preferably the support element penetrates the body of the expandable body. Even so the expandable body 2 has movement freedom relative to the support element. The reinforcement layer limits the movement of the expandable body. In one embodiment the reinforcement layer 9 limits the sliding movements of the expandable body 2. In another embodiment the reinforcement layer limits the twisting movements of the expandable body 2. In another embodiment the reinforcement layer limits the deflection movements of the expandable body. In another embodiment the reinforcement layer 9 alters the indentation movements (responses) of the expandable body.

In one embodiment the expandable body 2 is activated to expand from its delivery configuration to its treatment configuration in the presence of bodily fluid. Said expansion is triggered upon deployment of said expansion body 2 from a delivery catheter 15.

FIG. 3 illustrates the hydrogel 2, the wire 4, and the outer braid 9.

The braid 9 provides an outer structure. The outer braid 9 forms a protective shell around the hydrogel 2. Preferably to outer braid is made from a strong biocompatible fiber 18. Either a polymer or a metallic material is preferred as the fiber material. Examples of suitable fibers include PET, PTFE, UHMWPE, PEEK, PEN, Nitinol, Stainless steel. The fiber 18 may be monofilament or multifilament. A monofilament fiber is preferred.

The hydrogel is expandable in blood. The hydrogel is biostable and provides an outer non-thrombogenic surface. The hydrogel may be porous to promote expansion/healing. The hydrogel may be processed with a phase inversion process to create a micro porous structure.

The braid 9 may be of any fibre for example polystyrene, PTFE, Nitinol, stainless steel. Nitinol would allow the shape of the braid 9 to be set. The outer braid 9 may be manufactured using a number of techniques including braiding, knitting, and weaving. In one embodiment the braid 9 is fixed to the shaft 3. The outer braid may be fixed in a number of ways using conventional techniques. It may be integrally fixed at one or both ends. It may be slidable fixed at one or both ends. It may be fixed at one or both ends in a manner that allows it to rotate about the wire 4.

In use, the treatment element 2 in the storage configuration, the support element 4 and the anchor element 8 are housed in the delivery catheter 15. The delivery catheter 15 is then inserted into the patient's vasculature and advanced through the vasculature until the distal end of the delivery catheter 15 reaches the atrium 6. The support element 4 is advanced out of the delivery catheter through the atrium 6, through the valve opening 7, and into the ventricle 5 until the treatment element 2 is located at the region of co-aptation of the valve leaflets 3. The support element 4 is then rotated to screw the anchor element 8 into the ventricle wall at the apex of the ventricle 5. The treatment element 2 is thus supported in the desired location to treat the valve (FIG. 1).

When the treatment element 2 contacts the blood, the treatment element 2 expands from the storage configuration to the first treatment configuration. Upon contraction of the heart, the valve leaflets 3 engage with the treatment element 2 to move the treatment element 2 from the first treatment configuration to the second treatment configuration (FIG. 3). Upon relaxation of the heart, the valve leaflets 3 move away from the treatment element 2, and thus enable the treatment element 2 to move from the second treatment configuration back to the first treatment configuration.

If it is desired to remove the device 1, the support element 4 is rotated to unscrew the anchor element 8 from the ventricle wall. The support element 4 is then withdrawn from the ventricle 5 through the valve opening 7, and withdrawn from the atrium 6.

In FIGS. 4 to 7 there is illustrated another medical device 10 according to the invention, which is similar to the medical device 1 of FIGS. 1 to 3, and similar elements in FIGS. 4 to 7 are assigned the same reference numerals.

In this case the treatment element 2 comprises a plurality of beads of hydrogel material. The beads of hydrogel material are housed within the braid 9. The pores of braid 9 are smaller than the diameter of the beads. The pores if outer braid 9 are smaller than the beads of hydrogel when the beads of hydrogel are in the de-hydrated collapsed state.

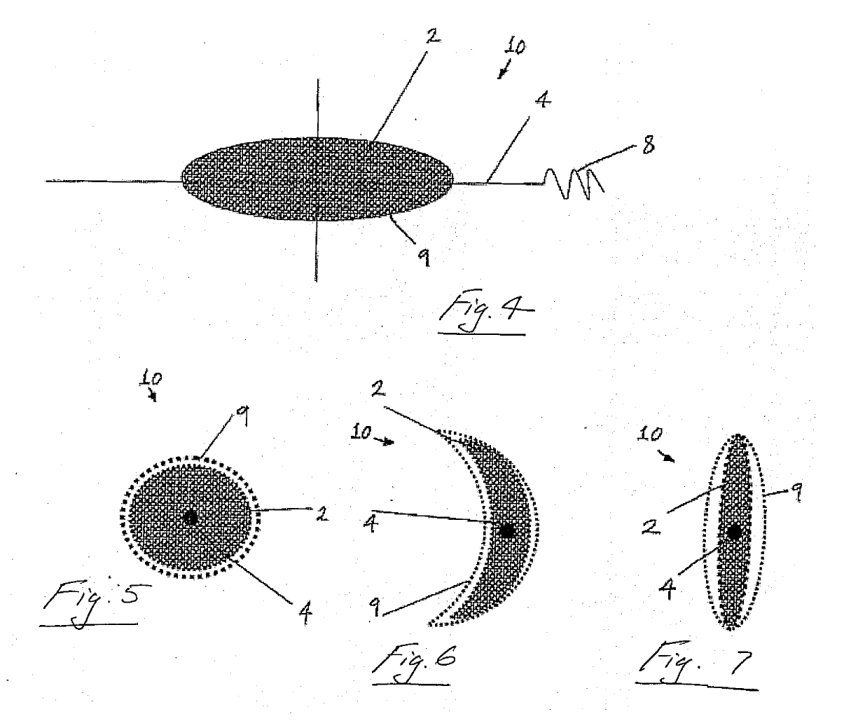

In the second treatment configuration, the treatment element 2 may have the crescent-shape in lateral cross-section (FIG. 6), or alternatively may have an oval-shape in lateral cross-section (FIG. 7). The shape of the treatment element in the second treatment configuration is determined by the forces of the valve leaflets. The hydrogel beads have a similar density to that of the surrounding blood and the beads can slide and move relative to each other within the outer braid 9. Therefore even small forces from the valve leaflets or blood flow can induce a shape change in the treatment element 2.

FIG. 4 illustrates the inner hydrogel beads 2, the wire 4, and the outer fabric structure 9.

FIG. 5 illustrates the relaxed state (diastole), FIG. 6 illustrates the deformed state (systole), and FIG. 7 illustrates an alternate state (systole). FIG. 5 illustrates the outer fabric structure 9, the wire 4, and the hydrogel particles/beads 2.

FIGS. 8 to 10 illustrate another medical device 20 according to the invention, which is similar to the medical device 10 of FIGS. 4 to 7, and similar elements in FIGS. 8 to 10 are assigned the same reference numerals.

In this case, the reinforcement element 9 extends around the full circumference of the treatment element 2 in the storage configuration. As the treatment element 2 expands from the storage configuration to the first treatment configuration (FIG. 9), the treatment element 2 expands through the reinforcement element 9. As a result, in the first treatment configuration, the reinforcement element 9 is embedded within the treatment element 2 (FIG. 9).

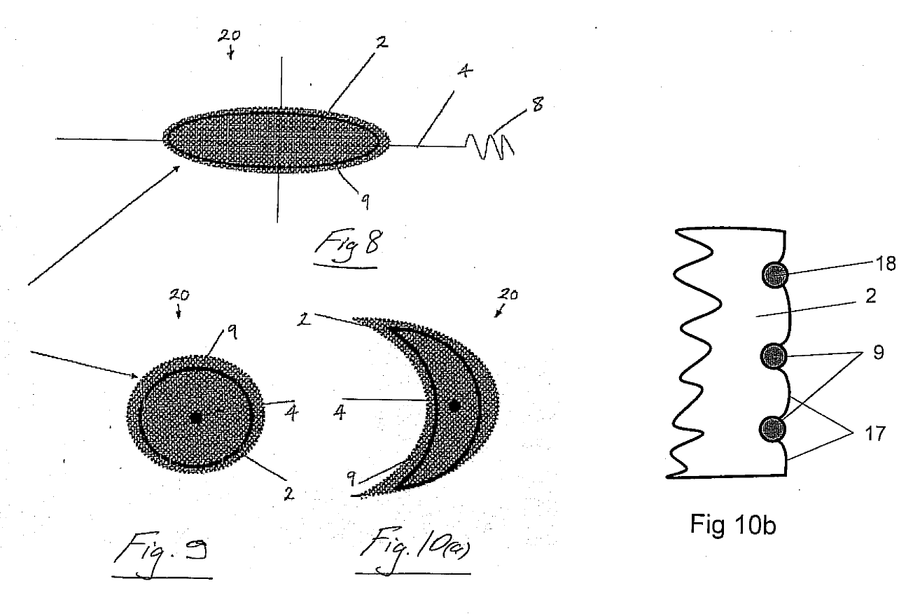

FIG. 8 illustrates the inner hydrogel beads 2 which expand through the fabric/braid 9, the wire 4, and the outer fabric structure 9. FIG. 9 illustrates the relaxed state (diastole), and FIG. 10 illustrates the deformed state (systole). FIG. 9 illustrates the hydrogel 2 bonded to RGD which encourages coverage of the device 20 with non-thrombogenic endothelial cells, the fabric structure 9, the wire 4, and the hydrogel particles 2 which expand through the fabric 9.

FIG. 10b shows a close up view of a section of the device of FIG. 10. The outer braid 9 is porous and in the expanded hydrated state the hydrogel surface 17 is exposed to blood through the pores of the outer braid 9. Preferably the fiber 18 is very small in diameter and preferably the total surface area of fiber 18 in contact with blood is less than the surface area of the hydrogel 9 in contact with blood in the expanded state. Ideally the area of fiber 18 in contact with blood is small relative to the area of hydrogel 9 in contact with blood.

In one embodiment the outer braid 9 is slightly imbedded in the surface of the hydrogel in the expanded state (FIG. 10b). These features mean that even though the hydrogel 9 is the inner member, that it still forms the primary blood interface of the device.

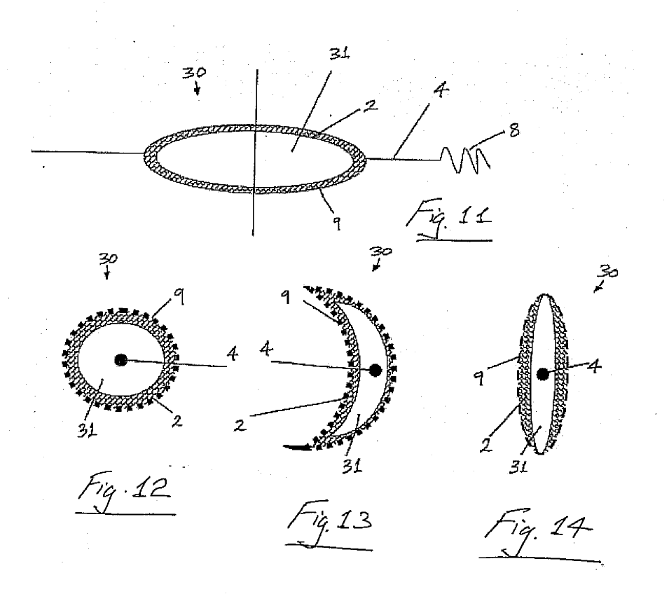

Referring to FIGS. 11 to 14 there is illustrated another medical device 30 according to the invention, which is similar to the medical device 1 of FIGS. 1 to 3, and similar elements in FIGS. 11 to 14 are assigned the same reference numerals.

In this case the treatment element 2 comprises a hollow interior space 31 which is enclosed.

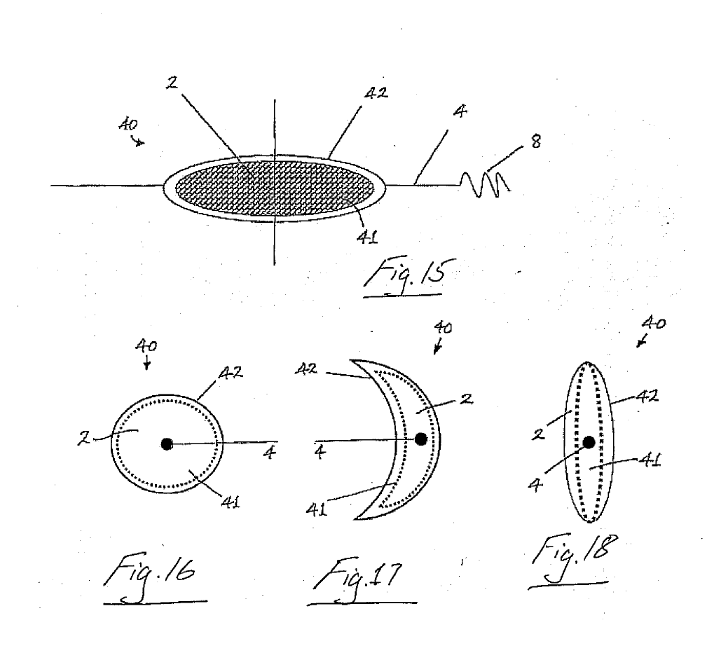

In the second treatment configuration, the treatment element 2 may have the crescent-shape in lateral cross-section (FIG. 13), or alternatively may have the oval-shape in lateral cross-section (FIG. 14).

FIG. 11 illustrates the hollow hydrogel fabric 2, the wire 4, the anchor 8, and the outer braid 9. FIG. 12 illustrates the relaxed state (diastole), FIG. 13 illustrates the deformed state (systole), and FIG. 14 illustrates an alternate state (systole). FIG. 12 illustrates the hollow hydrogel 2, the wire 4, and the outer braid 9 which acts as a structural support. The fibres are strong, and may be made into shaped objects by knitting/weaving/braid. The hydrogel 2 may expand when wet to fill the space 31. Alternatively the space may be filled with an injection of liquid by the user. With this embodiment a luer is provided in shaft 4 for inflation and a port connects the lumen with the hollow core. This hollow core reduces the mass of polymer associated with the treatment element and makes the device more trackable during delivery.

In FIGS. 15 to 18 there is illustrated a further medical device 40 according to the invention, which is similar to the medical device 1 of FIGS. 1 to 3, and similar elements in FIGS. 15 to 18 are assigned the same reference numerals.

In this case the treatment element 2 comprises an inner body 41 of a shape memory material, such as Nitinol, and an outer cover 42. No reinforcement element is provided in this case.

In the second treatment configuration, the treatment element 2 may have the crescent-shape in lateral cross-section (FIG. 17), or alternatively may have the oval-shape in lateral cross section (FIG. 18).

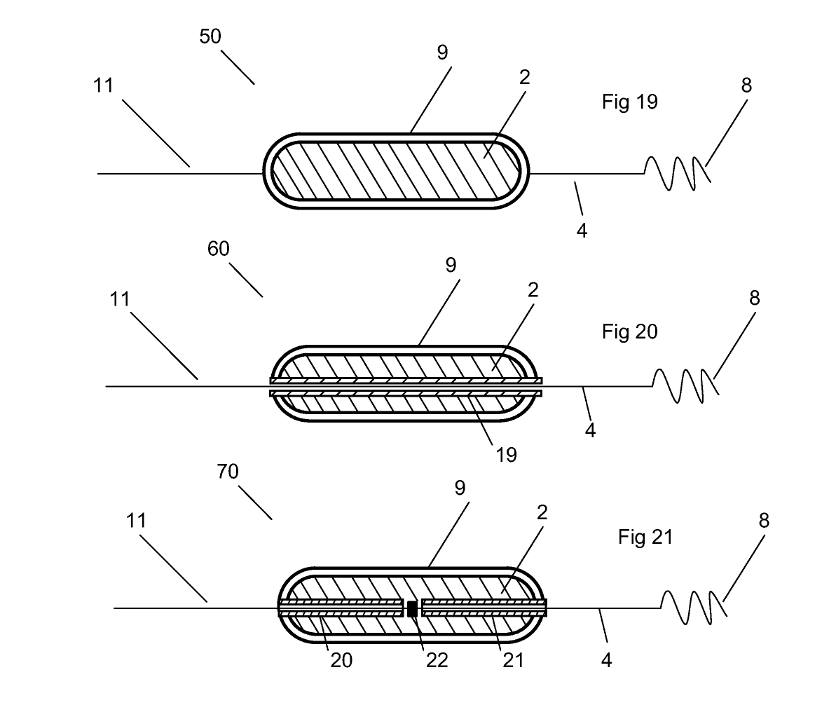

FIG. 15 illustrates the Nitinol braid 41, the soft covering 42, and the wire 4. FIG. 16 illustrates the relaxed state (diastole), FIG. 17 illustrates the deformed state (systole), and FIG. 18 illustrates an alternate state (systole). FIG. 16 illustrates the soft covering 42, the wire 4, a Nitinol braid or knitted Nitinol 41. The covering 42 may be a membrane for example polyurethane or silicone or polyurethane silicone, or a fabric for example a graft, polyester, PTFE, polyethylene, and may be either woven or knitted.

The covering may also be a hydrogel. The covering may be a solid or porous hydrogel. It may be knitted, braided or weaved from a hydrogel fiber.

FIG. 19 to FIG. 30 show how the treatment elements described in FIG. 1 to FIG. 18 are connected to the treatment wire 4 and the anchor 8. It will be appreciated that the mounting features described could be applied to any of the previous embodiments. Indeed the mounting features described here could also be applied to treatment elements other than those described herein. FIG. 19 shows a treatment element 2 and outer braid 9 directly mounted onto support element 4. This is similar to FIG. 2 except that the treatment 2 has a more elongated shape. It will be appreciated that a variety of cross-sectional shapes are possible. In this case the support element 4 comprises an anchor wire 11 and the anchor wire extends proximally of treatment element 2. The support element extends through the body of treatment element 2.

In another embodiment the reinforcement element 9 comprises a membrane. With this embodiment the reinforcement membrane 9 comprises the blood contact surface and the hydrogel 2 defines the shape of the treatment element in the expanded state.

In one variant the reinforcement membrane 9 is porous. Preferably the membrane pores are extremely small. The membrane pores are preferably smaller than blood cells. In another variant a lumen in the support element is used to transfer hydrating liquid to the hydrogel 2. Polymers with good biostability properties are preferred materials for the membrane. Silicone polymers, polyurethane polymers, polyolefin polymers, and fluoropolymers are suitable materials for the membrane. Polyether based polyurethanes and silicone based polyurethanes are especially suited as reinforcement membranes 9.

Now with reference to FIG. 20 there is shown another device 60. In this case the treatment element 2 is mounted on a mounting tube 19. The mounting tube 19 extends at least partially the length of the treatment element. The mounting tube comprises an inner lumen and an outer surface. The inner lumen is sized to accommodate the support wire 4. Alternatively the inner lumen is sized to accommodate the anchor wire 11. In one embodiment the inner lumen is a clearance fit with the support wire. In another embodiment the inner lumen is an interference fit with at least a portion of the support wire. In another embodiment the mounting tube 19 is fixed to the treatment wire.

FIG. 21 shows a device 70 wherein the mounting arrangement comprises a proximal mounting tube 20, a distal mounting tube 21 and a movement stop 22. The proximal mounting tube 20 comprises a lumen, an outer surface and a distal surface. The distal mounting tube 21 also comprises a lumen, an outer surface and a proximal surface. The treatment element 2 is mounted on the proximal 21 and distal 22 mounting tubes. The movement stop 22 is mounted on the support element 4. The movement stop 22 prevents the treatment element 2 from moving axially with proximal and distal engagement surfaces. The proximal and distal engagement surfaces of the movement stop 22 engage with the proximal and distal surfaces of the mounting tubes 21 & 22 and prevent axial movement of the treatment element relative to the support wire. It will be appreciated that some movement is possible when the distance between the distal and proximal surfaces of the mounting tubes 21/20 is greater than the length of the movement stop 22. The diameter of the movement stop 22 is preferably small relative to the diameter of the treatment element 2. Preferably the diameter of the movement stop 22 is as small as or smaller than the diameter of the mounting tube 21. Preferably the diameter of the movement stop 22 is larger than the inner diameter of the mounting tube 21. With this embodiment the anchor 8 can be rotated without rotating the treatment element 2. This allows the device 70 to be anchored before the treatment element 2 is expanded. Furthermore the treatment element 2 can rotate on the support element 4 after expansion and so can self centre in the regurgitant orifice. With this feature the treatment element 2 can adopt rotationally in response to forces applied to its surface from the valve leaflets 3. The treatment element thus self adjusts to find the optimum orientation for leaflet coaptation. This allows the treatment element to correct for placement errors. In another embodiment the outer braid 9 is fixed to the mounting tubes 21/20 at the distal end or the proximal end of the treatment element 2 or at both ends of the treatment element 2.

It will be appreciated that abutment stops in contact with blood are potential sites of thrombus formation. It is therefore an intention of this invention to make abutment stops exceedingly small so as to overcome this problem. This problem has been further overcome per this invention by placing the abutment stop within the body of the treatment element.

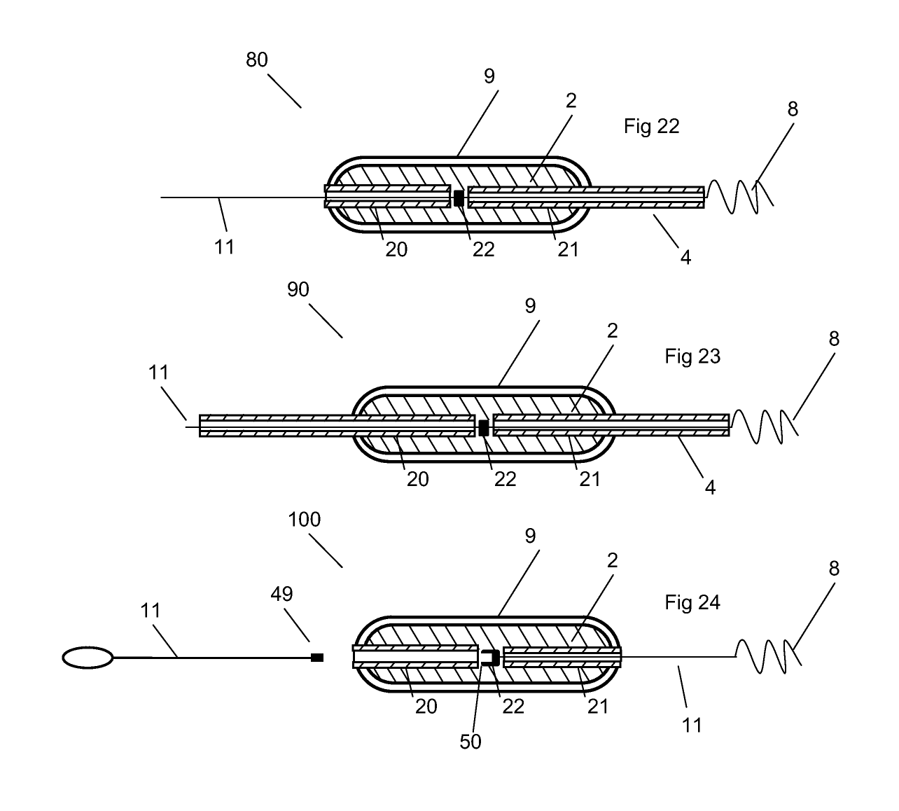

The device 80 of FIG. 22 is similar to device 70 and has similar features. In this embodiment the distal mounting tube 21 extends distal of the treatment element 2. In one embodiment the mounting tube extends from the movement stop 22 to the anchor 8. The mounting tubes 21/20 may be made from a number of materials and constructions. Preferred materials are biocompatible polymers and metals. In one embodiment the mounting tube is a polyurethane, a PEEK, a fluoropolymer, an olefin, a poly acrylate (PMMA), a polyester, a silicone polymer, a stainless steel alloy, nitinol, a shape memory metal, a super elastic metal or an alloy or copolymer of the above. The mounting tube may be an extruded tube, a hypotube, spring, or a coiled component.

The device 90 of FIG. 23 is similar to the devices 80 and 70 above. In this case the proximal mounting tube 20 extends proximal of the treatment element 2. In another embodiment of the invention the anchor wire 11 extends from the treatment element 2 proximally to the user. In one case the anchor wire is implanted with the treatment element 2 the support element 4 and the anchor 8.

In another embodiment at least a portion of the anchor wire 11 is detached from the implantable elements of the device including the treatment element 2. In order to facilitate detachment of a portion of the anchor wire the anchor wire and the implantable elements including the treatment element have features that facilitate coupling and decoupling 46. Coupling and decoupling features 46 may include threads, push fits, taper locks, snap fits, rotational engagements, rotational disengagements, axial engagements, axial disengagements and combinations of these. In one variation the anchor wire 11 is coupled to the treatment element 2 within the body of the treatment element 2. In another variation the anchor wire 11 is coupled to the treatment element 2 at the proximal or distal end of the treatment element 2. In another variation the anchor wire 11 is coupled to the mounting tube 20. In another variation the anchor wire 11 is coupled to a coupling 46 and said coupling 46 is integral with the treatment element 2 or the anchor 8. In one embodiment the coupling 46 is a collar, in another the coupling 46 is a mounting tube and in another the coupling 46 is a machined or moulded component. In yet another variation the anchor wire 11 is coupled with the anchor 8 at a point proximal of the distal end of the treatment element 2. Irrespective which of the above variations are employed the anchor wire 11 is decoupled and removed from the patient after device delivery. Preferably decoupling occurs after anchoring of the anchor 8 in the wall of the ventricle, after the treatment element 2 has been expanded at the treatment site and after the user has verified using either fluoroscopy of echocardiography that regurgitation has been improved.

FIG. 24 shows a device 100, similar to devices 80, 90, 100 and especially 70. With this device the proximal segment anchor wire 11 has been decoupled from the implantable elements. In this embodiment the implantable elements include the treatment element, the anchor wire and the anchor. In the embodiment shown the implantable elements also include the mounting tubes 20, 21, the movement stop 22, the outer braid 9 and the hydrogel 2. In this case the anchor wire 11 further comprises a coupling feature. The coupling feature comprises two coupling elements which engage or couple the proximal and distal segments of the anchor wire 11. The proximal segment of the anchor wire 11 has a first coupling element 49 near its distal end. The distal segment of the anchor wire 11 has the second coupling element 50 near its proximal end. A variety of coupling and decoupling mechanisms are possible. The coupling may be a mechanical coupling, an adhesive coupling, solder based coupling, a weld based coupling or other coupling means known in the art. Decoupling may include mechanical decoupling, solubilisation of coupling materials, electrolytic degradation of a coupling joint, or other decoupling means known in the art. Mechanical coupling is preferred due to its ease of use. A variety of mechanical couplings are possible. Mechanical couplings wherein the size of the coupling is close to the size of the anchor wire are preferred. One coupling element involves a first coupling element 49 wherein said coupling element comprises a male element and a second coupling element 50 wherein said coupling element comprises a female element. The male and female coupling elements engage to couple the proximal and distal end of the anchor wire 11. In one embodiment the male and female elements are tapered and from a taper lock. In another the male coupling element comprises at least one flat surface and the female coupling element comprises at least one flat surface and the engagement of said at least one flat surfaces couples the proximal and distal ends of the anchor wire in torsion.

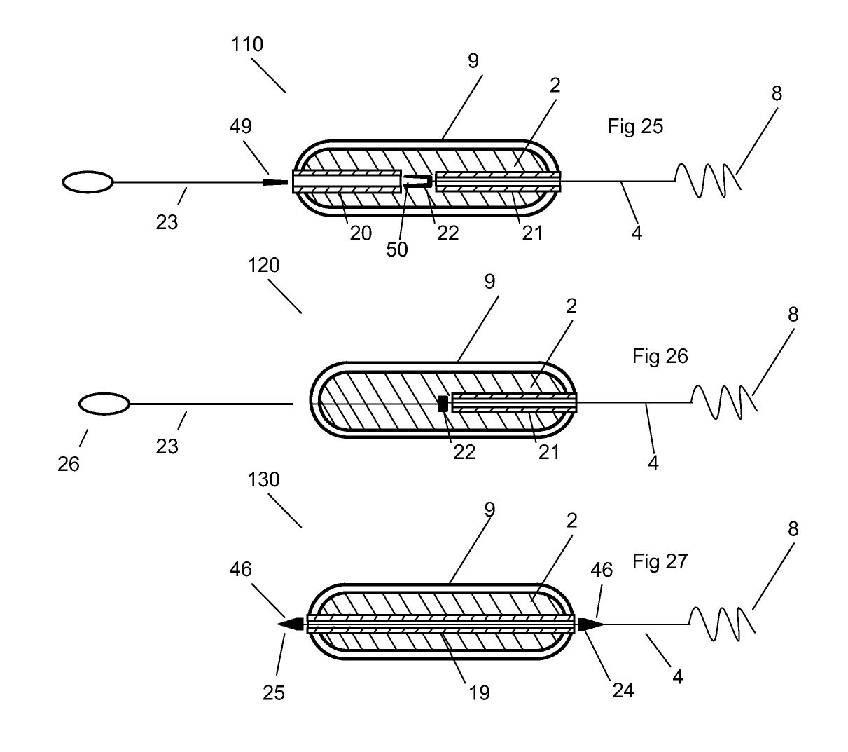

The position of the coupling feature along the length of the anchor wire is variable. In one embodiment the coupling feature lies proximal of the treatment element. In the embodiment shown in FIG. 24 and FIG. 25 the coupling feature lies within the body of the treatment element. Indeed in these examples the female portion 50 of the coupling element also comprises a mounting stop 22. In another embodiment the coupling feature lies distal of the treatment element 2. In yet another embodiment the coupling feature lies adjacent the anchor 11. With this embodiment a separate support element 4 is required if the anchor wire 11 is to be decoupled after treatment element 2 deployment. In this situation the anchor wire 11 passes through a lumen in the support element 4 and the support element 4 is connected to the anchor 11.

The coupling features described above allow the treatment element 2 and the anchor 8 to be advanced by pushing the anchor wire 11. Rotation of the anchor wire 11 by the user causes the male coupling element 49 to engage torsionally with the female coupling element 50 and said user torsion is transmitted to the anchor 8. Both of these movements facilitate anchoring the anchor 8 in the wall of the ventricle 5. When anchoring is complete the anchor wire 11 can be decoupled by retracting it proximally. Re-coupling is also possible by advancing and rotating the anchor wire 11. Preferably the anchor wire 11 comprises a high modulus material. Ideally the anchor wire 11 comprises a stainless steel, nitinol, a superelastic alloy, MP35N, L605 or other similar alloys. Alternatively the anchor wire 11 is a composite. In another embodiment the anchor wire 11 comprises a least partially a spring component.

With reference to FIG. 25 there is shown a device 110 which is very similar to device 100 of FIG. 24. In this case the anchor wire 11 comprises a torque handle 26. The torque handle allows the user to more easily and accurately rotate the anchor 11 and this provides better control of the anchoring step. The anchor wire 11 is shown decoupled from the implantable elements.

In one embodiment the distal mounting tube 24 and/or the proximal mounting tube 25 comprise a collar wherein said collar(s) encircle the support element and are fixed to a portion of the treatment element. In one variant the collar(s) is fixed to the treatment element 2. In another variant the collar is fixed to the reinforcement element 9. In another variant a portion of the reinforcement element 9 is configured into a collar(s). Said collar is small relative to the diameter of the treatment element. Preferably the diameter of the collar is only slightly larger than the diameter of the support element. Suitable dimensions of the collar outer diameter would be in the range:

Collar Outside Diameter=Support element OD+0.002″

Collar Outside Diameter=Support element OD+0.010″

Collar dimensions outside this range on the lower and upper end are possible and this invention is not limited by these dimensions.

FIG. 26 shows another device 120 and again this device is similar to previous devices. In this case there is only one mounting tube 21 and this mounting tube is positioned towards one end of the treatment element 2. In this case there is no mounting tube on the proximal side. The anchor wire in the delivery configuration passes through the treatment element on the proximal side. Where the treatment element 2 is a hydrogel the hydrogel is in intimate contact with the anchor wire 11. When the device is expanded and the anchor wire is removed the hydrogel swelling caused it to fill the lumen left by the removal of the anchor wire 11. This healing effect of the hydrogel is very beneficial as it prevents blood components clotting at the lumen entrance. With this embodiment the movement stop engages with the mounting tube 21 on one side and with the treatment element 2 on the other.

With reference to FIG. 27 there is shown another device 130 which is similar to previous devices. In this case one mounting tube 19 is used and this mounting tube extends at least a portion of the length of the treatment element. In one embodiment the ends of the mounting tube are adjacent the ends of the treatment element. In another the ends of the mounting tubes are within the boundaries of the treatment element. In another the ends of the mounting tube 19 extend beyond the ends of the treatment element 2. The device further comprises a proximal movement stop 25 and a distal movement stop 26. The movement stops comprise engagement faces and these engagement faces abut the treatment element. Preferably the engagement faces of the distal 24 and proximal movement stops 25 abut the distal and proximal surfaces of the mounting tube 19. Preferably the movement stops comprise a transition surface 46. The transition surface 46 is shaped so as to create a smooth interface between the movement stops 24, 25 and the neighbouring elements. Depending on the construction of the device, the neighbouring elements may include some or all of; the treatment element 2, the anchor wire 11, the mounting tube 19 and the support element 4. This smooth interface prevents the formation of clots on the surface of the device.

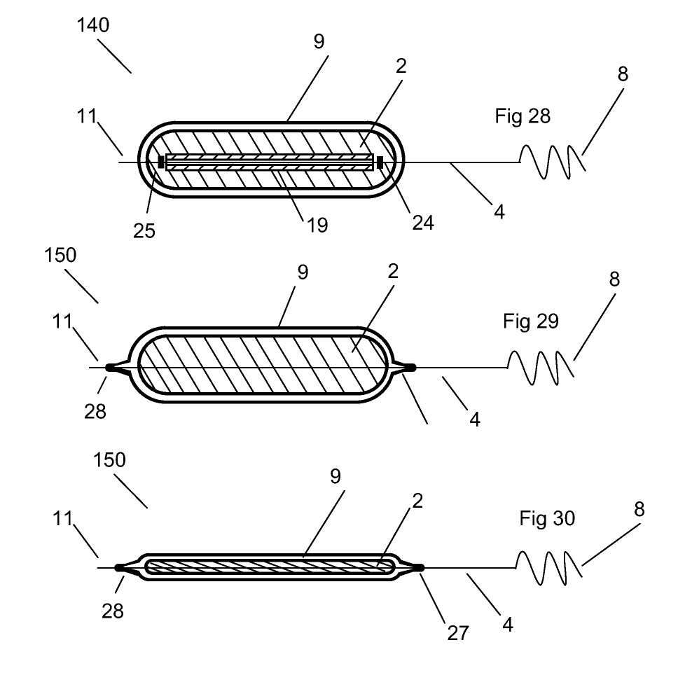

With reference to FIG. 28 there is shown another device 140 which is similar to previous devices. In this case the proximal stop 25 and the distal stop 24 are positioned within the body of the treatment element 2.

The device 150 of FIG. 29 is similar to the previous devices. In this case the treatment element is coupled to the support element 4 by the reinforcement element 9. The support element 4 passes through the body of the treatment element 2. In one embodiment no substantial attachment or bond exists between the treatment element 2 and the support element 4. With this embodiment the treatment element 2 can slide on the support element 4 and can rotate relative to the support element 4. The treatment element 2 is held in position at the treatment site by the reinforcement element 9. The reinforcement element 9 is coupled to the support element 4. The reinforcement element 9 is coupled to the support element 4 using either a proximal coupling element 28 and/or a distal coupling element 27. The reinforcement element 9 may be coupled so as to prevent rotational movement or axial movement or both. At least one end will be coupled so as to prevent axial movement. The coupled reinforcement element prevents substantial axial movement of the treatment element. Where the reinforcement element is coupled so as to prevent axial and rotational movement of the reinforcement element it will be appreciated that this will not necessarily completely prevent then the treatment element from movement. The treatment element 2 in one embodiment is not rigidly fixed to the reinforcement element and thus is free to move within the reinforcement element 9. Furthermore the reinforcement element may be a knitted element and thus allows for shape change even if the ends are rigidly fixed.

With reference to FIG. 30 there is shown the device 150 this time in the collapsed state. In the collapsed state the hydrogel 2 is dehydrated and the device assumes a low profile delivery configuration.

Now with reference to FIG. 31 to FIG. 35 there is shown a series of delivery catheters 15 per this invention. It will be appreciated that the delivery catheters 15 described herein could be used with any of the earlier devices. FIG. 31 shows a delivery catheter 15 which comprises a distal section 33 and a proximal end 34. A housing 32 at the distal end 33 comprises a reception space 43. The reception space 43 comprises a lumen and said lumen is sized to house at least the treatment element 2 in the collapsed state. In one embodiment the delivery catheter distal end 33 comprises a first diameter and the delivery catheter proximal end 34 comprises a second diameter wherein the first diameter is larger than the second diameter. A transition section 38 connects the first diameter and the second diameter. The delivery catheter 15 further comprises a split line 35. The split line 35 comprises a weakened line running along at least a portion of the length of the delivery catheter 15 and said split line 35 can be split so as to allow the removal of the delivery catheter 15. A notch 36 is provided at a proximal end of the split line to facilitate the initiation of split line rupture by the user. In one embodiment the delivery catheter 15 comprises one or more split lines. Preferably between 1 and two split lines 35 are employed.

The delivery catheter 15 comprises at least one lumen. Said lumen extends from the delivery catheter distal end 33 to the proximal end 34. During delivery the treatment element 2 sits inside the reception space 43 and the support element 4 extends proximally through a lumen of the catheter 15 and extends proximally of the proximal end 34 of the catheter 15.

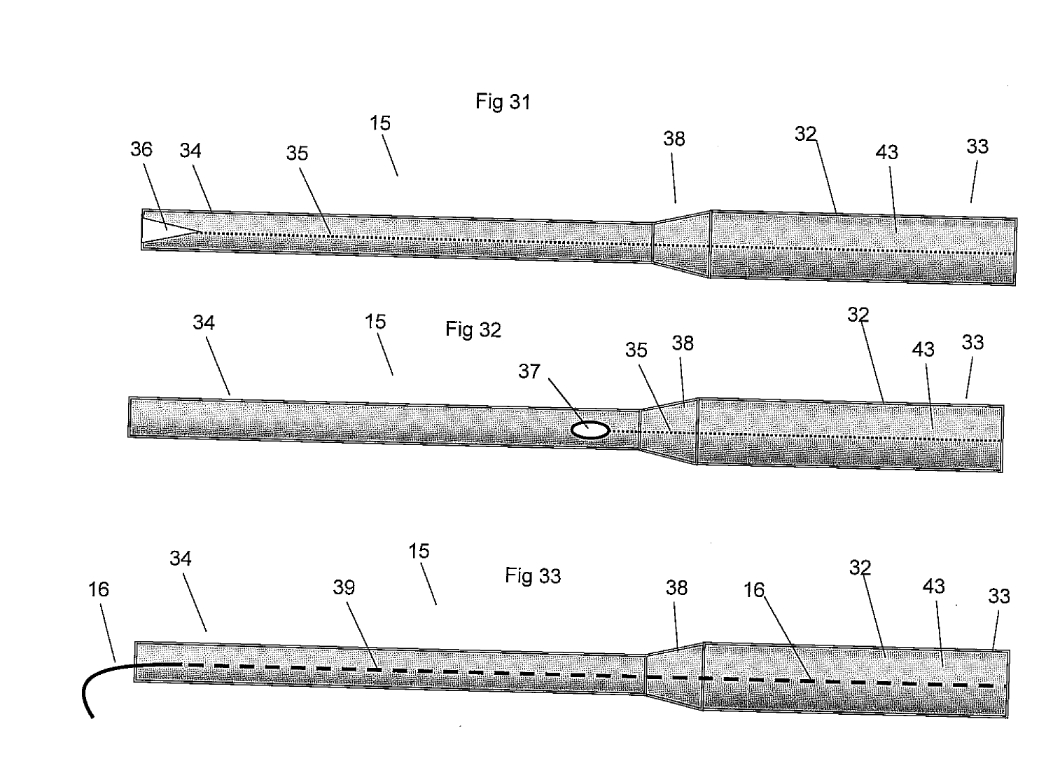

Now with reference to FIG. 32 there is shown another delivery catheter 15 which is similar to the delivery catheter 15 of FIG. 31. This time the delivery catheter 15 has an exit port 37 proximal of the reception space 43. The exit port is sized to allow the shaft of the support element to fit through the opening. With this device the split line 35 extends from the distal end of the catheter 15 to the exit port 37. In one embodiment the exit port is proximal of the reception space. In another embodiment the exit port is located in the transition region 38. The proximal section of the catheter 34 may be solid or it may comprise a flushing lumen. In one embodiment the delivery catheter 15 is supplied to the doctor with the proximal section of the support element 4 already extending through the exit port 37. With this embodiment the treatment element 2 and anchor 8 sit inside the reception space 43 and the support element 4 extends through the exit port 37. The delivery catheter 15 is advanced to the treatment site. The anchor 8 is anchored to a wall of the ventricle 5. The treatment element 2 is deployed from the delivery catheter reception space 43 and reduces or eliminates regurgitation. The final imaging is completed using echocardiography. The delivery catheter 15 is advanced proximally until the exit port 37 exits the patient. A rupture of the split line 35 is initiated by the doctor and the delivery catheter 15 is removed from the support element 4. The split line feature is especially important where a proximal hub is fixed to the proximal end of the support element 4. A notching accessory may be employed to initiate the rupture along the split line 35.

Now with reference to FIG. 33 additional features of the delivery catheters 15 of the invention are highlighted. The delivery catheter 15 may possesses some or all of the features described above, however steering features of the delivery catheter 15 are highlighted in this figure. A pull cable 16 is used to control the shape of the distal end 33 of the catheter 15. The pull cable 16 runs axially along one wall of the catheter 15. The proximal end of the pull cable can be activated by the user. In one embodiment the activation of the pull cable comprises a handle and thumbscrew. In another the activation comprises a lever. Preferably the pull cable 16 extends through a lumen in the wall of the catheter 15 and is fixed to the catheter wall near the distal end of the catheter 15. The distal end 33 of the catheter 15 is softer than the proximal end 34 and thus when the pull cable 16 is tensioned the distal end 33 of the catheter 15 assumes a curved shape. This allows the delivery catheter 15 to be steered to the treatment location. In one embodiment the steering cable is integral with the delivery catheter 15. In another embodiment the steering cable 16 is integral with the procedural sheath or guide catheter. In another embodiment more than one pull cable 16 is used to provide for more complex curves or to facilitate positive and negative curves.

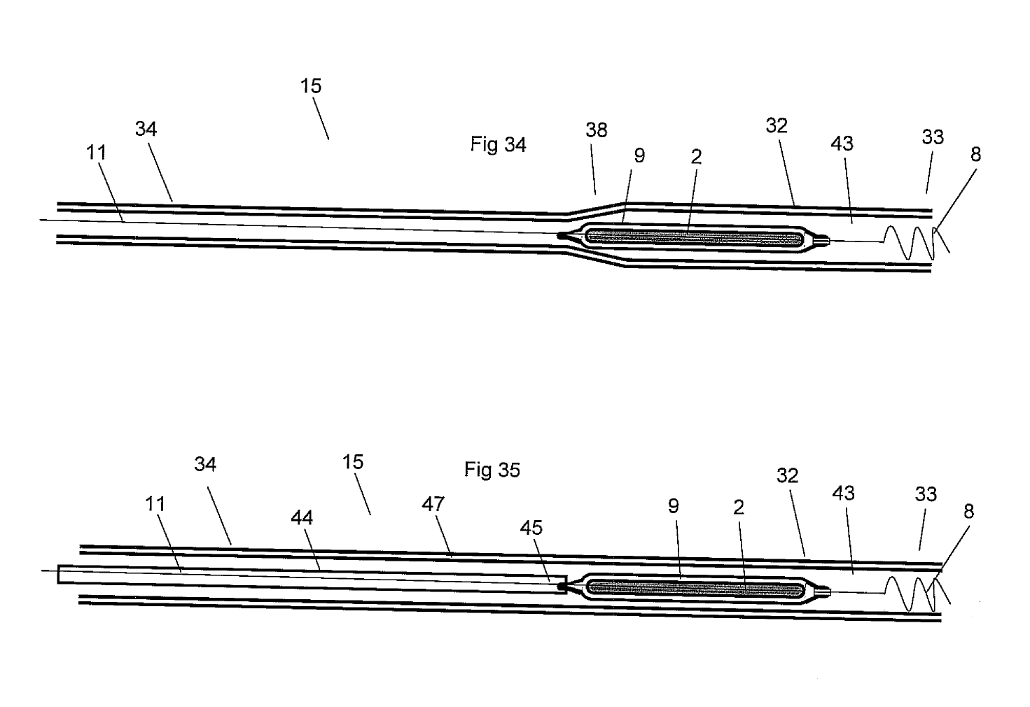

FIG. 34 shows a cross section of a delivery catheter 15 with a treatment element, support element 4 and anchor 8 located inside reception space 43. The treatment element 2 is in the collapsed configuration. The delivery catheter is advanced through the anatomy in this configuration. At the treatment location the anchor 8 is anchored to the ventricle wall. The delivery catheter 15 is retracted proximally while holding the anchor element 11 fixed. The anchor element 11 applies a force to the treatment element and the treatment element 2 is deployed.

FIG. 35 shows another cross section of a delivery catheter 15. This time the delivery catheter 15 comprises an outer catheter 47 and an inner shaft 44. The inner catheter 44 is moveable relative to the outer catheter 47 and is engagable with the proximal end of the treatment element 2. The treatment element 2 sits within the reception space 43 during delivery. At the treatment site the anchor 8 is anchored as described previously. In this case the inner shaft 44 can be used to deploy the treatment element 2. With this design it is possible to decouple the anchor wire 11 after the anchoring step and still deploy the treatment element 2 from the delivery catheter 15. Advancing the inner shaft 44 relative to the outer catheter 47 causes the treatment element to be deployed.

After the anchor 8 is anchored in the wall of the ventricle the cardiac cycle causes the position of the anchor 8 to move constantly in sync with the movements of the wall of the ventricle 5. If the anchor 8 fails to move with the movements of the wall of the ventricle 5 then a force is exerted on the anchor site 48. Excessive force at the anchor site 48 could cause the anchor 8 to pull out of the wall of the ventricle 5. It is therefore desired that the position of the treatment element 2 relative to the wall of the ventricle 5 be kept fairly constant to avoid applying unnecessary force to the anchor site 48. In order to keep this relative positioning of the treatment element 2 and the anchor site 48 the treatment element 2 must move in sync with the cardiac cycle. Otherwise compressive or tension forces will be applied to the anchor site 48 with the risk of the anchor 8 becoming dislodged from the anchor site 48. When the user is manipulating the device there is significant potential for the user to inadvertently apply excessive force to the anchor site 48. This is especially the case during the steps associated with treatment element deployment where elements of the device and delivery catheter 15 are moving relative to each other.

In order to avoid the anchor 8 becoming dislodged from the anchor site 48, the following method is employed during the deployment of the treatment element; The steps of device delivery and anchoring are described elsewhere in this specification.

With the anchor 8 anchored to the wall of the ventricle 5, the proximal end of the catheter is advanced slightly so as to create a slack segment in the catheter 15.

Confirm using imaging techniques that sufficient but not too much slack exists in the catheter 15.

Holding the inner shaft 45 stationary at the user end 34, advance the outer catheter 47 proximally until the treatment element 2 is deployed.

Removing the delivery catheter 15 from the patient.

Removing the proximal end of the support element 4 from the patient.

Removing of at least a portion of the anchor wire 11 from the patient.

This method allows the forces of deployment to be separated from the forces associated with cardiac cycle movements and therefore the movements associated with deployment are isolated from the cardiac movements and not transmitted to the anchor 11.

In one embodiment the catheter 15 has a slack promoting segment. This slack promoting segment is relatively softer than neighbouring segments and thus will bend more easily to take up slack. In one embodiment the slack segment is proximal of the distal end 33 of the catheter 15. In another embodiment the slack segment comprises the transition segment 38 of the catheter 15. In another embodiment the slack segment comprises the distal end of the proximal section 34 of the delivery catheter 15. The slack segment is preferably in a heart chamber and more preferably in the atrium 6. This slack will typically be evident by the presence of a curve in the catheter 15 in the slack segment and the curve will change during the cardiac cycle.

In another embodiment the delivery system comprises an elongate member and said elongate member comprises a slack promoting segment. The slack promoting segment is proximal of the distal end of the delivery system. Preferably the slack promoting segment is proximal of the treatment element 2 during delivery. In one embodiment the elongate member comprises an outer catheter 15 and the slack promoting segment comprises a relatively more flexible region of the catheter 15 proximal of the distal end. In another embodiment the elongate member comprises an outer catheter 15 and an inner shaft 44 and the slack promoting region comprises a relatively more flexible region of at least one of the outer catheter 15 or the inner shaft 44. Preferably the slack promoting region is proximal of the treatment element during delivery.

In another embodiment the elongate member comprises a support element and the support element 4 comprises a slack promoting region. In another embodiment the elongate member comprises an anchor wire 11 and the anchor wire 11 comprises a slack promoting region.

With reference again to FIG. 35 the inner shaft 44 is held relatively stationary during deployment and the outer catheter 47 is advanced proximally to effect deployment. It will be appreciated that with the other devices and delivery catheters 15 described in this invention that the support element 4 or the anchor wire 11 could be used to effect deployment and in these situations these elements are held relatively stationary during deployment and the outer catheter 47 advanced proximally to effect deployment. These elements could be used as replacement to the inner shaft 45 in the method described above.

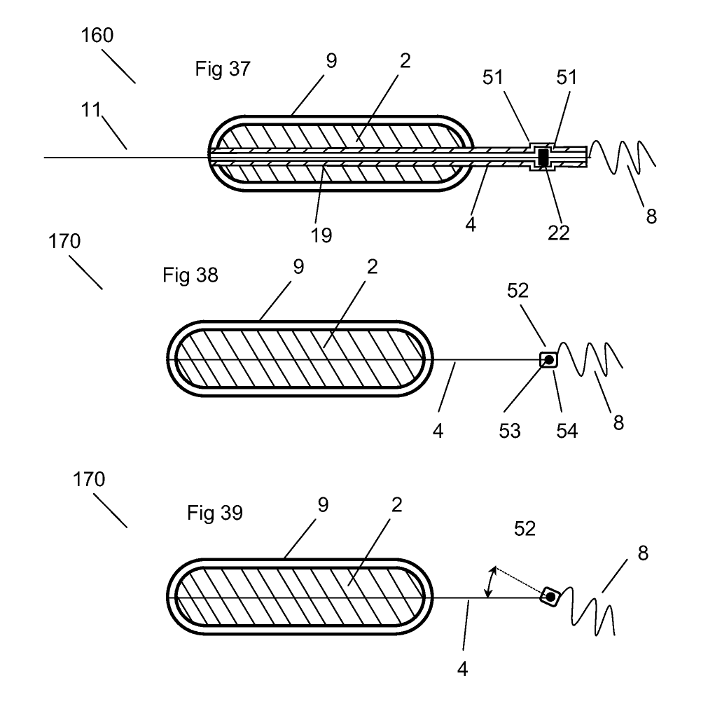

Referring to FIG. 37 there is shown another device 160 which is similar.

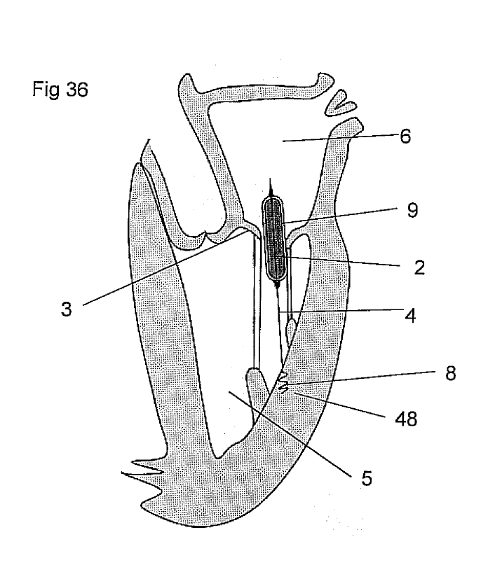

Now with reference to FIG. 36 there is shown one device of this invention in position within the ventricle. The anchor 8 is anchored to the wall of the ventricle 5. The support element connects the treatment element 2 to the anchor 8. The treatment sits within the mitral valve and the valve leaflets 3 coapt against its outer surface. The reinforcement element 9 provides additional integrity to the treatment element 2 at the treatment site. With the embodiment shown the proximal end of the treatment element 2 extends into the atrium. The treatment element 2 has a smooth profiled geometry throughout. The support element 4 terminates within the atrium 6. It will be appreciated that with other embodiments described that the support element 4 could extent at least partially back towards the user access site.

Referring to FIG. 37 there is shown a device 160, which is similar to previous devices. In this case, the treatment element 2 is mounted on the support 4. In use, the support 4 holds the treatment element 2 at the region of coaptation due to its association with the anchor 8. The support 4 comprises a recessed zone and said recessed zone comprises two abutment surfaces 51. The anchor 8 is fixed to the anchor wire 11 and the anchor wire 11 has an abutment stop proximal of its distal end. The abutment stop 22 is engageable with the two abutment surfaces 51 and this limits the movement of the treatment element 2 relative to the anchor 8. The movement of the anchor is controlled by the distance between the two abutment surfaces 51 and the length of the abutment stop 22. The anchor wire 11 extends proximally and allows the user to transmit torque and/or axial movements to the anchor 11. In one embodiment the proximal section of the anchor wire can be removed.

Referring to FIG. 38 there is shown a device 170, which is similar to previous devices. In this case, the treatment element 2 is mounted on support 4. In use, the support 4 holds the treatment element 2 at the region of coaptation due to its association with the anchor 8. The support 4 is associated with the anchor 8 via coupling 52. In one embodiment the coupling 52 is a hinged coupling. The hinged coupling 52 allows the treatment element to move in at least one arc about the hinge coupling 52. It will be appreciated that placing and implanting the anchor 8 in a beating heart is challenging and the final position of the anchor 8 will not be ideal in all cases. Because the treatment element 2 is free to rotate about the hinge point, the hinge coupling 52 prevents the anchor 8 from creating a bias in the support. Since the support 4 has no bias from anchor placement the invention allows the treatment element 2 to self centre in the valve 7 based primarily on forces of fluid flow, wall movement and leaflet interaction. Since the force required to deflect the hinge 52 is very low, the risk of the treatment element 2 corrupting the coaptation of a valve leaflet 3 will be reduced. In one embodiment the hinge coupling 52 comprises a universal joint. In another the hinge coupling 52 comprises an elbow style joint. In yet another embodiment the hinge coupling 52 comprises a ball and socket coupling. In yet another embodiment the hinge coupling 52 comprises an articulation region. The articulation region may be located in the support 4 or the anchor 8. With this embodiment the articulation region comprises a weakened section or a section of reduced bending stiffness. This may be achieved by thinning the support 4 or by flattening the support 4 or by connection two parts of the support 4 with a flexible material.

FIG. 39 shows the device 170 of FIG. 38 with the hinge 52 offset at an angle to the support 4. In one embodiment the hinge 52 is configured such that the offset angle is limited. Preferably the offset angle is limited at greater than 5 degrees. Preferably the offset angle is limited at greater than 10 degrees. Preferably the offset angle is limited at greater than 15 degrees. A hinge construction with a limited offset angle (see arrows) is achievable for example with a ball and flanged-collar arrangement. Where the hinge 52 is configured with a limited offset angle the support 4 (and treatment element 2) can hinge freely within a limited range. Where the hinge 52 has only one degree of freedom (elbow joint), then, the support 4 can move in a 2 dimensional arc with the hinge 52 as the centre of the arc. Where the hinge has two degrees of freedom as in a ball joint arrangement or a universal joint the support 4 (and treatment element 2) can move in an X-Y plane. With this embodiment the hinge 52 allows the treatment element 2 to move in two axes. This means the treatment element 2 can move along the line of coaptation of the valve leaflets or it can move at an angle to the line of coaptation of the valve leaflets. The ability of the treatment element to do this without needing to flex the support is a big advantage of this feature. It will be appreciated that in use the movement of the treatment element 2 about the hinge 52 will be significantly restricted by the boundaries of the valve 7 and the valve leaflets 3.

In one embodiment the support 4 comprises an anchor wire 11. With this embodiment the support 4 is used to advance and/or torque the treatment element in order to anchor the treatment element at the anchor site. Where the anchor comprises a screw in anchor, the, a torqueable hinge 52 such as a universal joint is preferred.

In another embodiment the support 4 further comprises a lumen and the anchor wire 11 extends from the anchor 8 proximally in the lumen of the support 4. The proximal end of the anchor wire 11 exits the patient and is configured to allow the user to implant the anchor in a tissue wall. In one variant at least a portion of the anchor wire can be decoupled after implanting the anchor 8 in a wall of body tissue as described earlier.

As described above the hinge 52 provides the treatment element 2 with two degrees of movement. It will be appreciated that this hinge feature can be combined with previously described embodiments to provide greater than two degrees of freedom to the treatment element 2. Where the treatment element 2 is slidable on the support 4 a third degree of freedom is possible. Where the treatment element is rotatable around the axis of the support 4 a fourth degree of freedom is possible. It will be generally appreciated that a variety of combinations of these degrees of freedom are possible.

In another embodiment while at least one of the movement freedoms described above is provided for, this at least one movement freedom is dampened. Dampening the movement freedom prevents an instantaneous response of the treatment element 2 to every force it experiences during the complicated cardiac cycle. This dampening of the movement freedom ensures that the treatment element 2 will move within a limited range of positions during the cardiac cycle. The position of the treatment element 2 at the point of coaptation at the beginning of systole is one key point in the cycle and it is desirable that the treatment element should not unnecessarily move a significant distance from this orientation at a less critical point in the cardiac cycle.

One approach to dampening the at least one movement freedom is to configure the moving elements such that there is some friction associated with the movements. This way the treatment element 2 will find a preferred (self centred) configuration over a series of cycles of the heart and friction will prevent excessive movements of the treatment element 2.

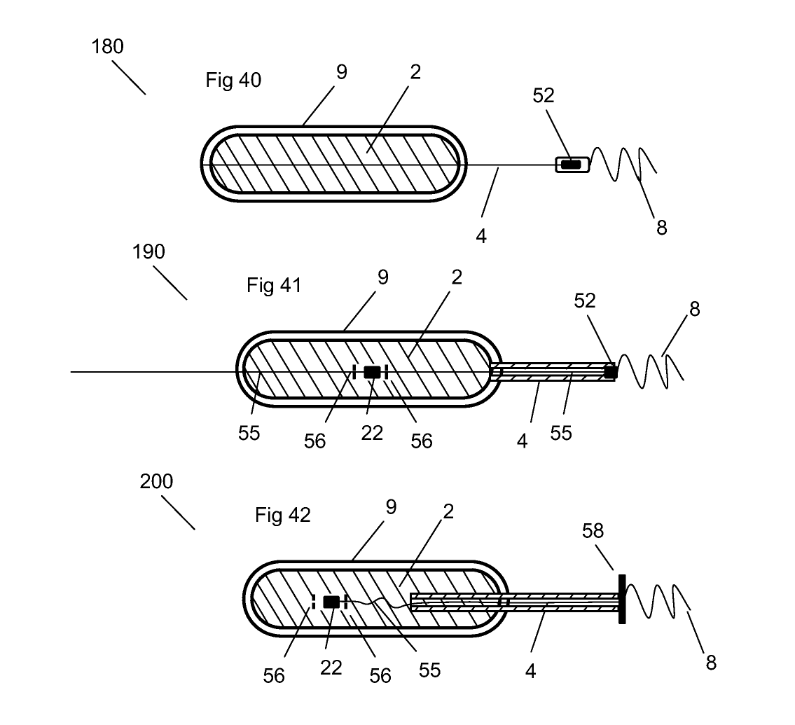

FIG. 40 shows a device 180 which is similar to devices previously described. In this case, the treatment element 2 is mounted on support 4. The support 4 holds the treatment element 2 at the region of coaptation due to its connection to the anchor 8. The support 4 is connected to the anchor 8 via coupling 52. Coupling 52 comprises a sliding arrangement. The sliding arrangement allows the support 4 to move axially relative to the anchor. The coupling comprises a collar with at least one flange and an abutment stop. The abutment stop engages with the flange to prevent further movement of the support. In one embodiment the coupling 52 is also hinged coupling. In another embodiment the coupling 52 comprises a housing and a stop. In this case the housing has at least one flat face on its inner surface and the stop has at least one flat face on its outer surface. Both faces are configured to interact when the anchor wire is rotated and this coupling arrangement allows torque to be transmitted to the anchor.

FIG. 41 shows a device 190 which is similar to devices previously described. In this case, the treatment element 2 is mounted on support 4. The support 4 holds the treatment element 2 at the region of coaptation due to its connection to the anchor 8. In this case the treatment element 2 can slide axially on support 4. The device further comprises a stop 22 and a tether 55. The stop is mounted on the tether 55 a fixed length from the anchor 8. The tether is fixed to the anchor at attachment point 52. The treatment element further comprises abutment surfaces 56. The abutment surfaces 56 engage with the abutment stop 22 and limit the movement of the abutment stop 22. The engagement surfaces are spaced apart and positioned within the body of the treatment element. In one embodiment the tether comprises a wire, a cable, a multifilament, or a monofilament.

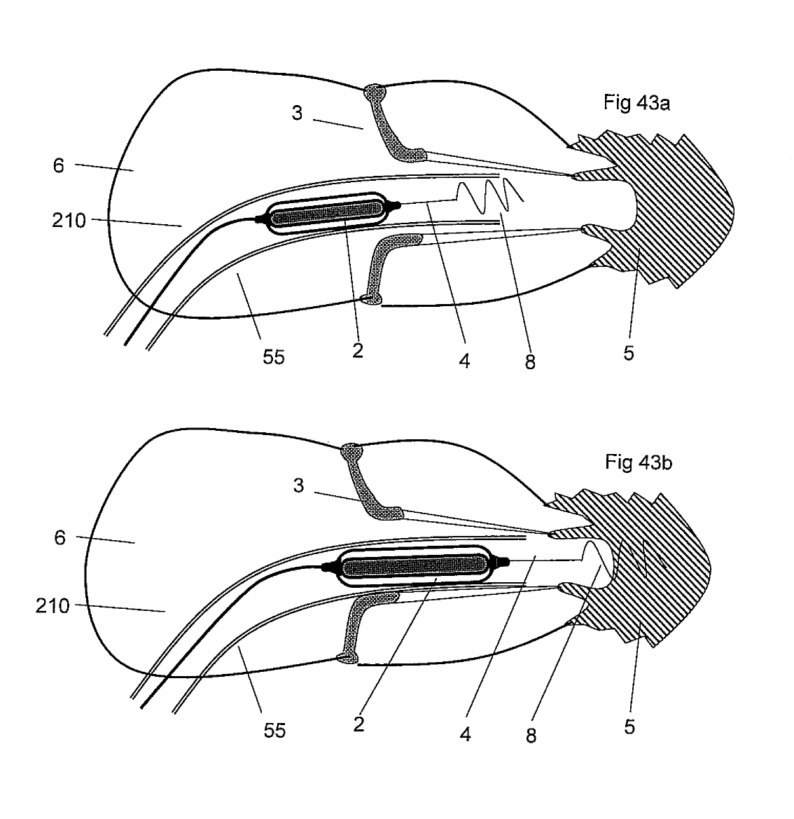

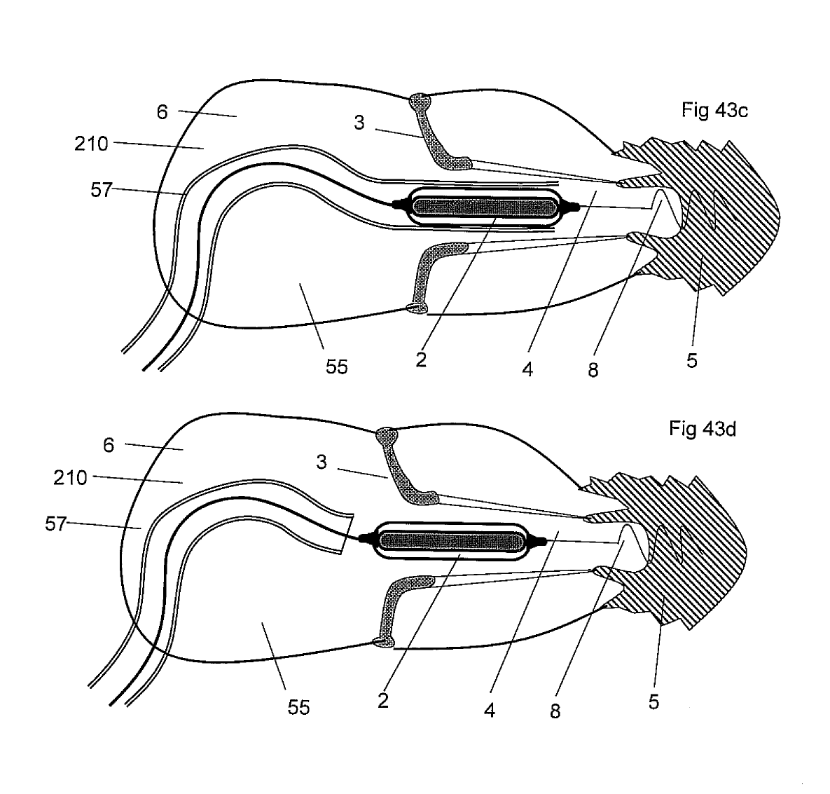

FIG. 42 shows a device 200 which is similar to devices previously described, especially FIG. 41. In this case, the tether is a flexible cable over at least a portion of its length. When the treatment is advanced distally along the support the tether offers no resistance as it has no significant compressive rigidity. In this case the movement of the treatment element 2 is limited by the support element 4. The support element 4 engages with an engagement surface 56 of the treatment element to limit axial movement.

Yet another embodiment of the invention is highlighted in FIG. 42. The anchor element 8 further comprises an anchor limiter 58. The anchor limiter 58 prevents the anchor 8 from continuing to penetrate the tissue wall. In so doing it prevents the anchor from penetrating through the wall tissue. In the case of a heart wall a perforation could have significant consequences. In one embodiment the limiter is expandable. In another the anchor limiter is deployed from a delivery device 57 as part of the anchoring step. In another embodiment the material of the anchor limiter comprises a polymer, a metal, a stainless steel, a nitinol, a shape memory material, a super elastic material, a stainless alloy. In another embodiment the anchor limiter is a nitinol element with an expanded state and a collapsed state. In another embodiment the anchor limiter 58 comprises arms extending radially outwardly in the expanded state. In another embodiment the anchor limiter 58 interacts with the tissue surface to reinforce the grip of the anchor 8. In another embodiment the anchor limiter 58 interacts with the tissue wall and prevents the anchor 8 from disengaging with the tissue wall. One variant of this feature involves the anchor limiter 58 preventing the anchor 8 from unscrewing from the tissue wall.

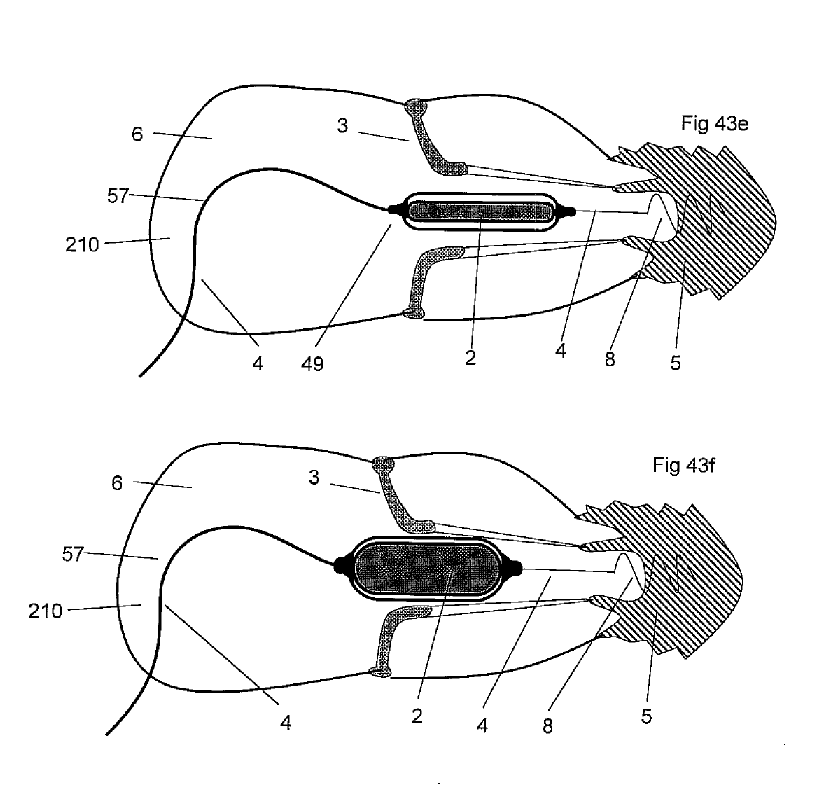

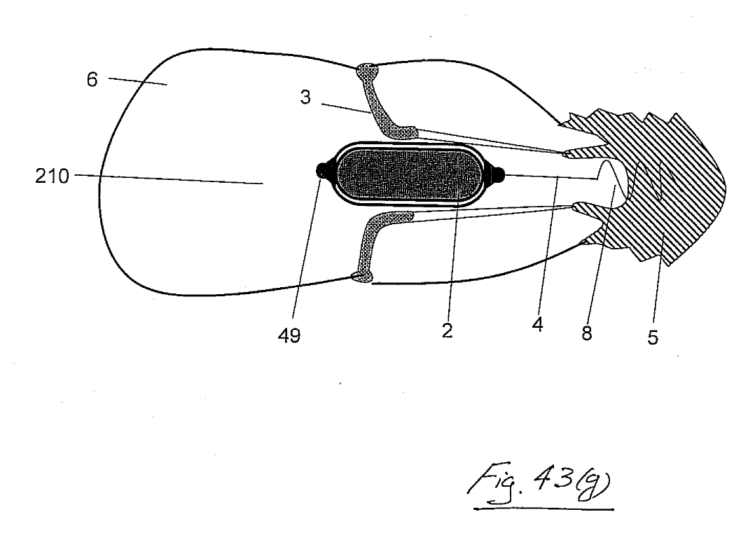

FIG. 43a to FIG. 43g show a series of steps that are employed with some of the devices of this invention. FIG. 43a shows a step in the method of delivery of a device 210 of the invention. With this embodiment the device 210 comprises a treatment element 2, an anchor 8, a support 4 and a delivery device 57. The anchor 8 is configured to be anchored to a wall of body tissue. The treatment element 2 has a collapsed state and an expanded state. In the figure the device 210 is advanced into a heart chamber. In this case the device is advanced into the atrium. This is achieved by the physician making an intra-atrial septal puncture. It will be appreciated that the device 210 could also be advanced into a heart chamber through the atrial wall with a surgical technique, through the aortic valve with an endovascular approach, through the apex of the ventricle with a surgical approach.

The device is advanced across the valve leaflets in the collapsed state. The device is preferably steered as it is advanced across the leaflets.

FIG. 43b shows another step in the procedure whereby the anchor 8 is anchored to a wall of body tissue. In the case shown the wall is the ventricle wall 5. In one embodiment, the anchoring step comprises the step of advancing the anchor 8 relative to the delivery device 57. In one embodiment, the anchoring step comprises the step of advancing the anchor 8 relative to the treatment element 2. Advancing the anchor is achieved by pushing the support while holding at least a portion of the delivery device 55 steadfast.

In another embodiment the support 4 comprises an inner shaft and an outer shaft and the anchoring step comprises moving the inner shaft relative to the outer shaft to anchor the anchor 8 in the tissue wall. With this construction the anchor 8 is expandable and the step of implanting the anchor 8 in the wall of body tissue comprises advancing the anchor 8 into a tissue wall 5 and expanding the anchor 8 within the tissue wall.

FIG. 43c and FIG. 43d show the steps associated with the deployment of the treatment element. After the anchor 8 has been anchored in a tissue wall, the delivery device is advanced. The delivery device comprises a slack promoting region. The slack promoting region is located in a heart chamber. In the embodiment shown the slack promoting region is proximal of the treatment element and is located in the atrium. It will be appreciated that the slack promoting region could be in the ventricle chamber or it could be distal of the treatment element.

The slack promoting region of the delivery device 55 causes the delivery device to take a curved shape in the heart chamber. This curved shape changes throughout the cardiac cycle as it accommodates heart wall movements. The procedure further comprises the step of imaging the delivery device 55 to ensure sufficient slack is in place to accommodate the wall movements of the cardiac cycle.

When sufficient slack is in the heart chamber the device can be deployed without applying pull out forces to the anchor. The step of deploying the treatment element involves retracting at least a portion of the delivery device 57 while holding the support 4 steadfast. In one embodiment the delivery device 57 comprises a delivery catheter with a reception space 43 at its distal end and the step of withdrawing the delivery catheter 57 comprises unsheathing the treatment element 2.

The procedure further comprises the step of removing at least a portion of the delivery device 57 from the patient. The step of expanding the treatment element 2 is depicted in FIG. 43f. In one embodiment the treatment element comprises a hydrogel and the delivery device covers the hydrogel during delivery. When the delivery device 55 is withdrawn the treatment element is exposed to blood and expands by absorbing fluids from the surrounding blood. With this embodiment the step of expanding the device comprises unsheathing the device and activating expansion by contact with bodily fluids.

In another embodiment the step of expanding the treatment element comprises inflating the treatment element with a biocompatible fluid. Suitable fluids include biocompatible liquids and gasses such as saline, and contrast media. With this embodiment the support element comprises a lumen in communication with the fluid space of the treatment element 2 and extending proximally to the user. Fluid is delivered through the lumen and at least partially fills the fluid sac. In another embodiment the treatment element 2 comprises a self expanding element and expands when the sheath of the delivery device 55 is retracted. With this embodiment the step of expanding the treatment element comprises retracting a restraining sheath 55 from the treatment element 2.

The procedure further comprises the step of sizing the treatment element 2 to the regurgitant orifice. In one embodiment this step comprises injecting a volume of fluid into a fluid sac of the treatment element 2 through a lumen in the support element.

With reference to FIG. 43g the procedure further comprises the step of decoupling at least a portion of the delivery device 57 from the support.

The procedure further comprises the step of decoupling at least a portion of the delivery device 57 from the treatment element.

The procedure further comprises the step of decoupling a proximal portion of the support 4 from the distal portion of the support 4.

The procedure further comprises the step of decoupling the anchor wire from the support 4.

The invention is not limited to the embodiments hereinbefore described, with reference to the accompanying drawings, which may be varied in construction and detail.

1-206. (canceled)

207. A heart valve implant comprising:
a spacer configured to interact with at least a portion of at least one cusp of a heart valve to at least partially restrict a flow of blood through said heart valve in a closed position;
a shaft having a first end configured to be coupled to said spacer; and
an anchor configured to be coupled to a second end of said shaft, said anchor configured to removably secure said implant to engage native cardiac tissue within a chamber of a heart.

208. The implant of claim 207, wherein said spacer is operative coupled to said anchor to provide a degree of movement with respect to said anchor to allow the spacer to self-align with respect with respect to said at least a portion of said at least one cusp of said a heart valve to at least partially restrict a flow of blood through said heart valve in said closed position.

209. The implant of claim 208, wherein said second end of said shaft is pivotally coupled to said anchor to provide said degree of movement.

210. The implant of claim 209, further comprising a gimbal pivotally coupling said second end of said shaft to said anchor.

211. The implant of claim 208, wherein said shaft is configured to bend to provide said degree of movement.

212. The implant of claim 207, wherein said spacer is configured to at least partially expand from a collapsed configuration wherein said spacer is configured to be received in and advanced along a lumen of a delivery catheter to an expanded configuration wherein said spacer is configured to interact with at least a portion of at least one cusp of a heart valve to at least partially restrict a flow of blood through said heart valve in a closed position.

213. The implant of claim 212, wherein said spacer comprises a resiliently flexible balloon configured expand from said collapsed configuration to said expanded configuration, wherein said resiliently flexible balloon is configured to interact with at least a portion of at least one cusp of a heart valve to at least partially restrict a flow of blood through said heart valve in a closed position.

214. The implant of claim 213, wherein said resiliently flexible balloon is disposed at least partially over a resiliently flexible cage, said resiliently flexible cage being coupled to said first end of said shaft.

215. The implant of claim 214, wherein said resiliently flexible cage further comprises a frame or ribbed structure configured to provide additional support to said resiliently flexible balloon.

216. The implant of claim 215, wherein said resiliently flexible cage comprises a plurality of support ribs extending generally along a longitudinal axis of said implant.

217. The implant of claim 216, wherein said plurality of rigs are configured to resiliently bend radially inwardly and outwardly.

218. The implant of claim 217, wherein said resiliently flexible cage comprises a shape memory material.

219. The implant of claim 212, wherein said spacer comprises a generally cylindrical cross-section.

220. The implant of claim 219, wherein said spacer further comprises a generally conically shaped region proximate a first and a second end region of said spacer.

221. The implant of claim 207, wherein said anchor comprises a base and a plurality of tines extending generally outwardly from said base, said plurality of tines configured to removable engage native cardiac tissue to removably secure said implant within said chamber of a heart.

222. The implant of claim 221, wherein said plurality of tines extend generally radially outwardly from said base towards said spacer.

223. The implant of claim 222, wherein said anchor comprises a generally inverted umbrella configuration.

224. The implant of claim 221, wherein said anchor is pivotally coupled to said second end of said shaft.

225. The implant of claim 207, wherein said implant further comprises at least one releasable coupler configured to releasably engage a delivery device configured to position said implant within said chamber of said heart.

226. A heart valve implant comprising:
a spacer configured to interact with at least a portion of at least one cusp of a heart valve to at least partially restrict a flow of blood through said heart valve in a closed position;
a shaft having a first end configured to be coupled to said spacer; and
an anchor configured to removably secure said implant to native cardiac tissue within a chamber of a heart;
wherein said spacer is operatively coupled to said anchor to provide a degree of movement with respect to the anchor to allow the spacer to self-align with respect to said at least a portion of said at least one cusp of said a heart valve to at least partially restrict a flow of blood through said heart valve in said closed position.

227. A heart valve implant comprising:
a spacer configured to interact with at least a portion of at least one cusp of a heart valve to at least partially restrict a flow of blood through said heart valve in a closed position;
a shaft having a first end configuration to be coupled to said spacer;
an anchor configured to removably secure said implant to native cardiac tissue within a chamber of a heart; and
a gimbal pivotally coupling said anchor to a second end of said shaft, said gimbal configured to provide a degree of movement with respect to said anchor to allow said spacer to self-align with respect to said anchor to allow said spacer to self-align with respect to said at least a portion of said at least one cusp of said a heart valve to at least partially restrict a flow of blood through said heart valve in said closed position

228. A heart valve implant comprising:
a spacer configured to interact with at least a portion of at least one cusp of a heart valve to at least partially restrict a flow of blood through said heart valve in a closed position;
a shaft having a first end configured to be coupled to said spacer; and
an anchor configured to be coupled to a second end of said spacer, said anchor comprising a base and a plurality of tines extending generally outwardly and away from said base, said plurality of tines configured to removably secure said implant to native cardiac tissue within a chamber of a heart.

229. The implant of claim 228, wherein said spacer is operatively coupled to said anchor to provide a degree of movement with respect to the anchor to allow the spacer to self-align with respect to said at least a portion of said at least one cusp of said a heart valve to at least partially restrict a flow of blood through said heart valve in said closed position.

230. The implant of claim 229, wherein said second end of said shaft is pivotally coupled to said anchor to provide said degree of movement.

231. The implant of claim 230, further comprising a gimbal pivotally coupling said second end of said shaft to said anchor.

232. The implant of claim 229, wherein said shaft is configured to bend to provide said degree of movement.
